(19) United States
(12) Patent Application Publication
(10) Pub. No.: US 2025/0302398 A1
(43) Pub. Date: Oct. 2, 2025

(54) ANALYTE MONITORING SYSTEMS

(71) Applicant: ABBOTT DIABETES CARE INC., Alameda, CA (US)

(72) Inventors: Swati Satish, Fremont, CA (US); Saranpreet S. Nagra, Alameda, CA (US); Sabine Kabel-Eckes, Mountain View, CA (US)

(21) Appl. No.: 19/004,143

(22) Filed: Dec. 27, 2024

Related U.S. Application Data
(60) Provisional application No. 63/616,584, filed on Dec. 30, 2023.

(51) Int. Cl. A61B 5/00; A61B 5/145; G16H 10/65; G16H 40/67

(52) U.S. Cl. CPC A61B 5/742; A61B 5/14532; G16H 10/65; G16H 40/67; A61B 2560/0487

(57) ABSTRACT

Improved graphical user and digital interfaces for analyte monitoring systems are provided. For example, disclosed herein are various embodiments of GUIs including, sensor results, trend alerts, alarms, and insights interfaces. In addition, various embodiments of digital interfaces are described, including methods for sensor activation and pairing, wherein an analyte monitoring software application is configured to pair with a plurality of different types of sensors, and methods data backfilling in an analyte monitoring system, among other embodiments.

### CROSS-REFERENCE TO RELATED APPLICATIONS

This application claims priority to U.S. Provisional Application No. 63/616,584, filed Dec. 30, 2023, which is herein expressly incorporated by reference in its entirety for all purposes.

### FIELD

The subject matter described herein relates generally to improvements to analyte monitoring systems, as well as computer-related methods and devices relating thereto.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Thus, needs exist for improved digital interfaces, graphical user interfaces, and software for analyte monitoring systems, as well as methods and devices relating thereto, that are robust, user-friendly, and provide for timely and actionable responses.

### SUMMARY

Provided herein are example embodiments of improvements to in vivo analyte monitoring systems. Improved graphical user and digital interfaces for analyte monitoring systems are provided. For example, disclosed herein are various embodiments of GUIs including, sensor results, trend alerts, alarms, and insights interfaces. In addition, various embodiments of digital interfaces are described, including methods for sensor activation and pairing, wherein an analyte monitoring software application is configured to pair with a plurality of different types of sensors, and methods of data backfilling in an analyte monitoring system, among other embodiments.

Many of the embodiments provided herein include improved GUIs or GUI features for analyte monitoring systems that are highly intuitive, user-friendly, and provide for rapid access to physiological information of a user. More specifically, these embodiments allow a user to easily navigate through and between different user interfaces that can quickly indicate to the user various physiological conditions and/or actionable responses, without requiring the user (or an HCP) to go through the arduous task of examining large volumes of analyte data. Furthermore, some of the GUIs and GUI features, such as the sensor usage interfaces, allow for users (and their caregivers) to better understand and improve their respective levels of engagement with their analyte monitoring systems. Likewise, many other embodiments provided herein comprise improved digital interfaces and/or features for analyte monitoring systems that improve upon: the accuracy and integrity of the analyte data being collected by the analyte monitoring system by allowing for data backfilling, flexibility of the analyte monitoring system by allowing users to transition between different reader devices, alarming functionality of the analyte monitoring system by providing for more robust inter-device communications during certain adverse conditions, to name only a few.

The improvements to the GUIs in the various aspects described and claimed herein produce a technical effect at least in that they assist the user of the device to operate the device more accurately, more efficiently and more safely. It will be appreciated that the information that is provided to the user on the GUI, the order in which that information is provided and the clarity with which that information is structured can have a significant effect on the way the user interacts with the system and the way the system operates. The GUI therefore guides the user in the technical task of operating the system to take the necessary readings and/or obtain information accurately and efficiently. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. Aspects of the embodiments are set out in the independent claims and preferred features are set out in the dependent claims. The preferred features of the dependent claims may be provided in combination in a single embodiment and preferred features of one aspect may be provided in conjunction with other aspects. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 3A through 3I are block diagrams depicting example embodiments of launch, onboarding and account GUIs, or features related thereto, for an analyte monitoring software application.

FIGS. 4A and 4B are block diagrams depicting example embodiments of tutorial and setting interfaces, or features related thereto, for an analyte monitoring software application.

FIG. 4C depicts a flow diagram of an example embodiment of a method for monitoring a nearby devices permission.

FIGS. 4D through 4F are block diagrams depicting example embodiments of tutorial and setting interfaces, or features related thereto, for an analyte monitoring software application.

FIG. 4G depicts a flow diagram of an example embodiment of a method for monitoring a wireless communication protocol permission.

FIGS. 4H and 4I are block diagrams depicting example embodiments of tutorial and setting interfaces, or features related thereto, for an analyte monitoring software application.

FIG. 5A-1 depicts a flow diagram of an example embodiment of a method for pairing a sensor with an analyte monitoring software application.

FIG. 5A-2 depicts a flow diagram of an example embodiment for a data backfilling method in an analyte monitoring system.

FIG. 5A-3 depicts a flow diagram of an example embodiment of a method for pairing a sensor with an analyte monitoring software application.

FIG. 5A-4 depicts a flow diagram of an example embodiment for a data backfilling method in an analyte monitoring system.

FIG. 5A-5 depicts a flow diagram of an example embodiment of a method for pairing a sensor with an analyte monitoring software application.

FIGS. 5B through 5E are block diagrams depicting example embodiments of sensor activation interfaces, or features related thereto, for an analyte monitoring software application.

FIGS. 5F through 5J are block diagrams depicting example embodiments of sensor results or home interfaces, or features related thereto, for an analyte monitoring software application.

FIGS. 6A through 6J depict block diagrams of example embodiments of profile and account interfaces, or features related thereto, for an analyte monitoring software application.

FIGS. 7A through 7M depict block diagrams of example embodiments of alarm interfaces, or features related thereto, for an analyte monitoring software application.

FIGS. 8A and 8B depict block diagrams of example embodiments of insights interfaces, or features related thereto, for an analyte monitoring software application.

In addition, attached hereto as Appendix A, and incorporated by reference for all purposes.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, software, and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

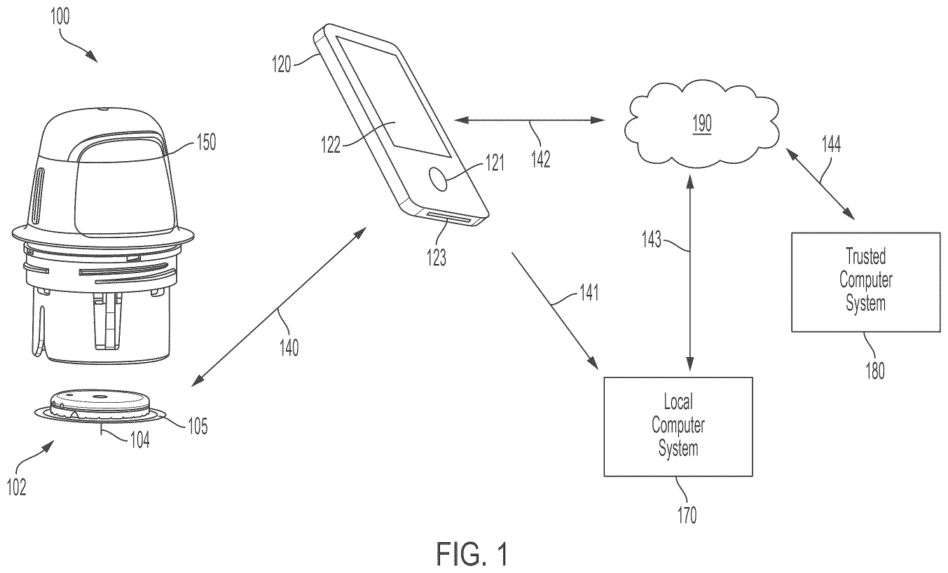

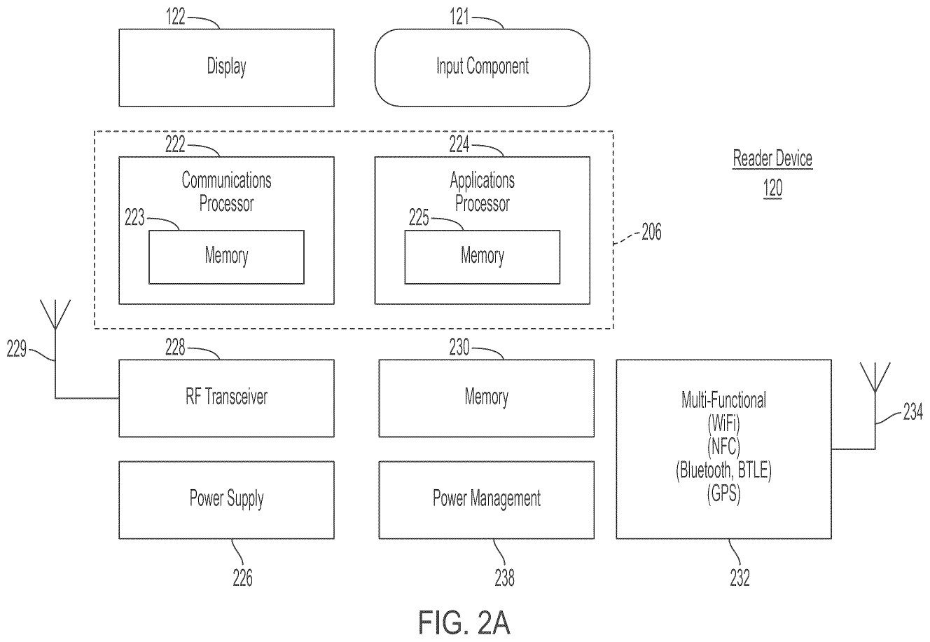

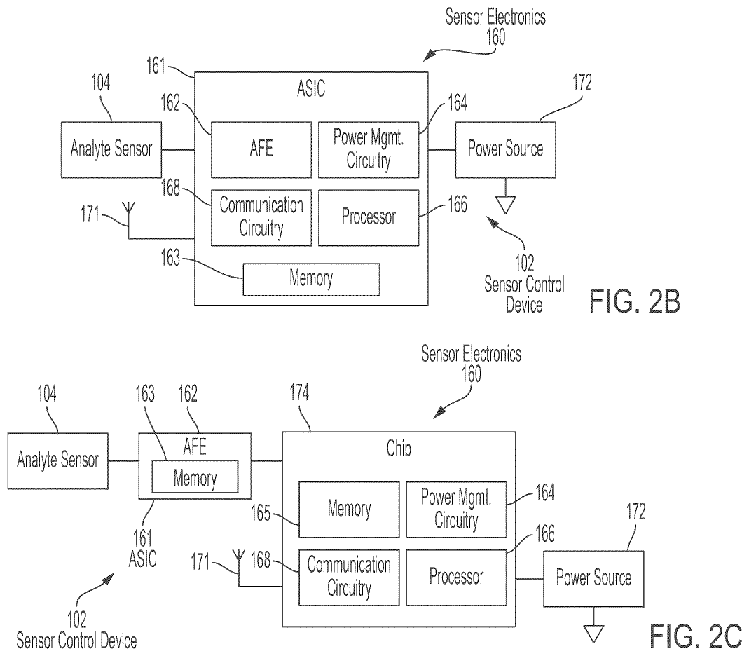

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth (BLE), Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical analyte (e.g., glucose, ketone, lactate) values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose and/or analyte value, historical glucose and/or other analyte values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Analyte Monitoring Software Applications

Described herein are example embodiments of an analyte monitoring software application (also herein referred to as an “App” or “glucose monitoring software application”) for analyte monitoring systems, as well as methods and systems relating thereto. As an initial matter, it will be understood by those of skill in the art that the graphical user interfaces (“GUIs”) and related methods described herein comprise instructions stored in a non-transitory memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the non-transitory memory of a single centralized device or, in the alternative, can be distributed across multiple discrete devices in geographically dispersed locations. It will be understood by those of skill in the art that any of the GUIs (or portions thereof) described herein, are meant to be illustrative only, and that the individual elements, or any combination of elements, depicted and/or described for a particular embodiment or figure are freely combinable with any other element, or any combination of other elements, depicted and/or described with respect to any of the other embodiments

Also described herein are example embodiments of alarms, alarm features, and alarm settings for analyte monitoring systems, as well as methods, systems, and GUIs relating thereto. As an initial matter, it will be understood by those of skill in the art that the alarms, alarm features, and alarm settings, as well as the related methods and interfaces described herein, can comprise instructions (e.g., software, firmware, etc.) stored in non-transitory memory of a sensor control device 102, reader device 120, local computer system 170, trusted computer system 180, and/or any other computing device or system (e.g., cloud-based server) that is part of, or in communication with, analyte monitoring system 100. These instructions, when executed by one or more processors of the corresponding system or device, cause the one or more processors to perform any or all of the method steps, and/or output the alarms or alarm interfaces described herein. Those of skill in the art will further recognize that the alarms, alarm features, alarm settings, and alarm interfaces described herein can be stored as instructions in the non-transitory memory of a single centralized device or, in the alternative, can be distributed across multiple discrete devices in geographically dispersed locations.

### Example Embodiments of Launch, Onboarding and Account GUIs and Features Related Thereto

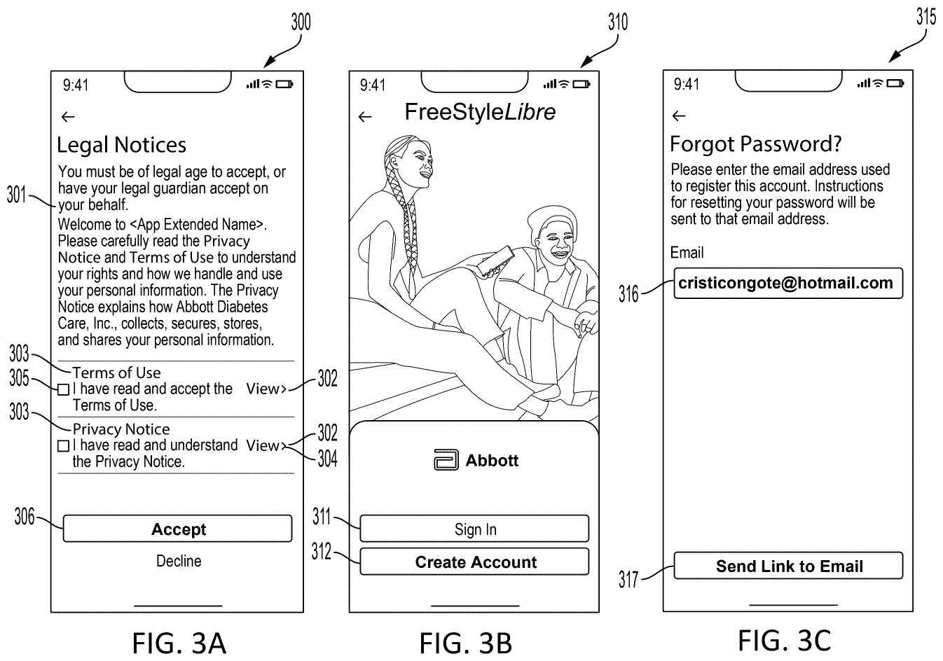

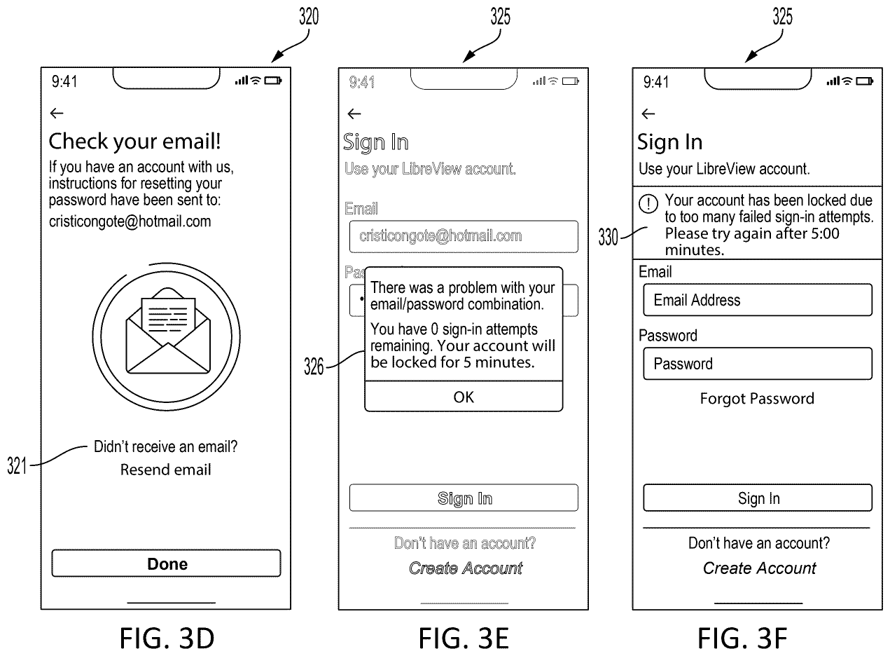

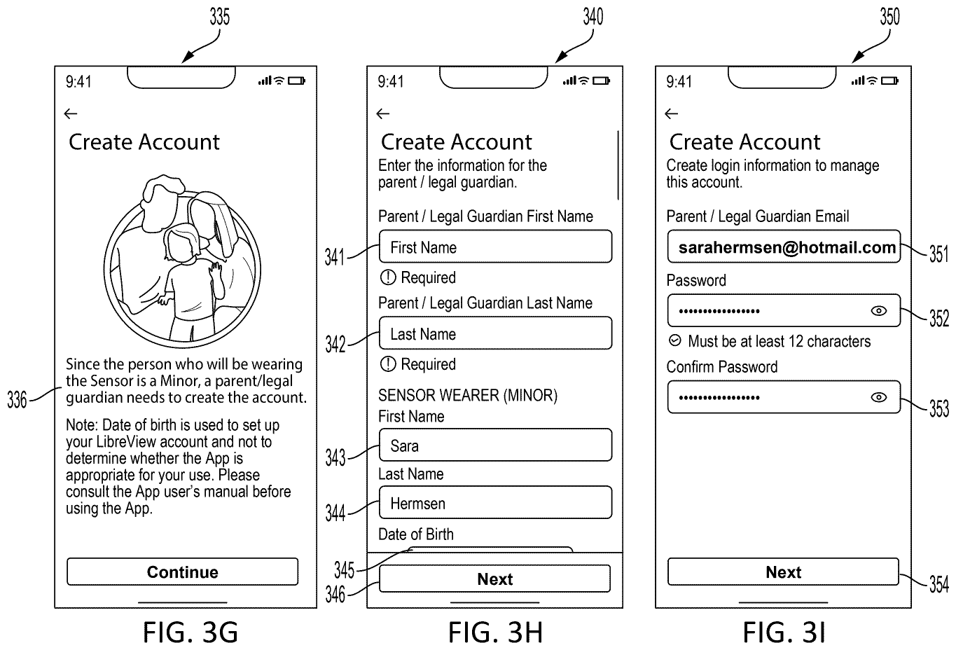

FIGS. 3A through 3I depict block diagrams of example embodiments of launch, onboarding and account GUIs, or features related thereto, any of which can be utilized with the analyte monitoring software application described herein. According to one aspect of the embodiments, the GUIs described herein are configured to display data and other health information through the software (e.g., the analyte monitoring software application) installed on a reader device, such as a smart phone or a receiver. Those of skill in the art will also appreciate that the software (e.g., analyte monitoring software application) with a GUI can also be implemented on a local computer system or other computing device (e.g., wearable computing devices, smart watches, tablet computer, etc.).

Example embodiments of methods, systems, and related GUIs for launching/onboarding of the analyte monitoring software application in an analyte monitoring system will now be described. According to one aspect of the embodiments, an analyte monitoring software application can be installed on reader device 120 (e.g., smart phone). After installing the analyte monitoring software application, the analyte monitoring software application can be launched on reader device 120. Specifically, in some embodiments, upon the user first opening the App on the reader device 120, a splash screen is displayed, then subsequently a series of launch or onboarding GUIs are outputted to the display. For example, in some embodiments, upon the user first opening the App and subsequent to the splash screen being displayed, the user is directed to read and acknowledge agreements and legal notices through a launch or onboarding GUI.

In particular, FIG. 3A is a block diagram depicting an example embodiment of a launch GUI 300 for an analyte monitoring system, wherein launch GUI 300 comprises information related to legal notices, such as, Terms of Use and a Privacy Notice. In some embodiments, launch GUI 300 comprises a message 301 indicating to the user that the user must be of legal age to accept the legal notices or have a legal guardian accept on the user's behalf. Further, according to an aspect of the embodiments, launch GUI 300 comprises a plurality of legal notice acceptance cards 302. Specifically, in some embodiments, each of the plurality of legal notice acceptance cards 302 can include (1) a textual description 303 indicating a legal notice represented by the respective legal notice acceptance card 302 (e.g., “Terms of Use” or “Privacy Notice”); (2) a selectable view link 304 which, upon being selected, outputs an interface comprising the legal notice represented by the respective legal notice acceptance card 302 (e.g., though not illustrated, a Terms of Use interface of a Privacy Notice interface can be outputted to the display); and (3) a selectable checkbox 305 which the user can select to indicate that the user has read and accepted the legal notice represented by the respective legal notice acceptance card 302. In some embodiments, the checkbox 305 is adjacent to a message stating that the user has read and understood the legal notice. Launch GUI 300 can further comprise a selectable accept button 306 and a selectable decline button so as to allow the user to accept or decline, respectively, the legal notices detailed in launch GUI 300.

According to an aspect of the embodiments, if the user selects the accept button 306 without selecting all checkboxes 305 displayed on launch GUI 300, then a pop-up modal (not illustrated) can be displayed indicating to the user that the user must accept all (e.g., both) notices by pressing or selecting both checkboxes in order to use the App.

Further, in some embodiments, though not illustrated, if, for example, a wireless communication protocol (e.g., a Wi-Fi connection) is interrupted, an error modal can be outputted to indicate that the there was an error loading the page and that the wireless communication protocol (e.g., internet connection) should be checked.

Example embodiments of methods, systems, and related GUIs for onboarding and compatibility checking for an analyte monitoring software application in an analyte monitoring system will now be described. According to some embodiments, various alarms and alarm features (which will be described in further detail below) can be associated with an analyte monitoring software application residing in memory of a reader device 120 (or other computing devices used in conjunction with an analyte monitoring system). Thus, in such embodiments, it would be advantageous to include certain onboarding and compatibility checking features during the setup of the analyte monitoring software application to ensure that the alarms and other features can operate as intended. In some embodiments, a compatibility check is performed each time the analyte monitoring software program is foregrounded.

In some embodiments, the analyte monitoring software application is configured to perform a compatibility check for both operating system compatibility and device compatibility. In some embodiments, the compatibility check comprises comparing the type and version of the reader device's operating system against a list, table, or database of compatible operating system types and versions stored on a remote computing system (e.g., cloud-based server). In some embodiments, if the remote computing system is inaccessible, then the analyte monitoring software application can use a list, table, or database stored locally on the reader device. In still other embodiments, the compatibility check can comprise transmitting the type and version of the reader device's operating system to the remote computing system. Subsequently, the remote computing system sends a response back to the reader device.

According to one aspect of some embodiments, the compatibility check can result in two or more outcomes. In many embodiments, for example, if it is determined that the operating system has been tested and is compatible with the analyte monitoring software application, then a compatibility GUI is displayed indicating that the operating system has been tested and is compatible with the analyte monitoring software application, and the analyte monitoring software application is functional with a predetermined set of features enabled.

According to some embodiments, if it is determined that the operating system type or version has not been tested for compatibility with the analyte monitoring software application, then a compatibility GUI is displayed indicating to the user that (1) the compatibility of the device and/or operating system with the App has not been confirmed along with a warning that certain features may not function correctly (e.g., “some App features, such as glucose alarms, may not be supported”); or (2) the user should update the App because a new App version is available along with a warning that certain features may not function correctly. In addition, in some embodiments where the operating system type or version has not been tested, certain features of the analyte monitoring software application may be disabled.

According to many embodiments, if it is determined that the operating system type or version is not compatible with the analyte monitoring software application, then a compatibility GUI is displayed, and a limited set of features are enabled for the analyte monitoring software application. For example, in some embodiments where the operating system type or version is not compatible with the analyte monitoring software application, then alarms can be disabled. In other embodiments, both alarms and sensor readings can be disabled, but the user can still review historical data or reports, if available. In still other embodiments, the entire analyte monitoring software application can be disabled. Specifically, in some if it is determined that the operating system type or version is not compatible with the analyte monitoring software application, then the user is prevented from progressing through subsequent onboarding and settings GUIs, and is further prevented from utilizing the analyte monitoring software application.

According to an aspect of some embodiments, the compatibility GUIs described herein can comprise information and an icon, link, or button to display a compatibility guide. According to one aspect of the embodiments, the compatibility guide can list all devices, operating systems, operating system types, and operating system versions that are confirmed to be compatible and/or incompatible with the analyte monitoring software application. In some embodiments, the compatibility guide can also provide information or instructions to the user about what to do if the analyte monitoring software application has been determined to be incompatible with the reader device and/or the operating system. In some embodiments, the analyte monitoring software application instructs the user to check the compatibility guide before updating the operating system or analyte monitoring software application on a new reader device.

Although the above compatibility check is described with respect to the reader device's operating system type and version, those of skill in the art will recognize that other aspects of hardware and software can be analyzed as part of the compatibility check, including but not limited to reader device model, reader device hardware componentry (e.g., minimum requirements for processor, memory, storage), other software applications installed on the same reader device, sensor control device type, sensor control device version, sensor control device model number, sensor control device firmware version, sensor control device hardware componentry, regional and/or geographical information, amongst others. This list is meant to be illustrative only, and those of skill in the art will appreciate that other factors regarding the compatibility of various software and/or hardware components of computing devices in an analyte monitoring system are fully within the scope of the present disclosure. Further details regarding compatibility checking and features related thereto are described in U.S. Publ. No. 2022/0248988, which is incorporated by reference in its entirety for all purposes.

According to yet another aspect of the embodiments if it is determined that the operating system has been tested and is compatible with the analyte monitoring software application, then a start GUI can be outputted to the display. Specifically, with reference to FIG. 3B, a block diagram is depicted of an example embodiment of a start GUI 310, wherein start GUI comprises (1) a selectable Sign In button 311 which, upon being selected, outputs a sign-in interface that allows the user to enter account credentials so as to sign into a cloud-based user account in order to utilize the analyte monitoring software application; and (2) a selectable Create Account button 312 which, upon being selected outputs an account creation interface which allows the user to initiate a process to create a cloud-based user account. According to one aspect of the embodiments, a cloud-based user account can allow the user to share information with healthcare professionals, family and friends; utilize a cloud-based reporting platform to review more sophisticated analyte reports; and back up the user's historical sensor readings to a cloud-based server.

In some embodiments, a sign-in interface (not illustrated) can comprise a selected forgot password button which the user can select if the password credentials associated with an existing cloud-based user account has been forgotten. Specifically, upon the user selecting the forgot password button, a password GUI 315 (FIG. 3C) can be outputted to the display. As shown in FIG. 3C, password GUI 315 can comprise a message requesting that the user enter the email address used to register the account and informing the user that instructions will be sent to the entered email address. Password GUI 315 can further comprise a text-entry field 316, wherein the user can manually enter the email address associated with the existing cloud-based user account, and a selectable confirmation link 317 which, upon being selected sends a link with instructions on resetting the password to the email address provided in the text-entry field 316.

In some embodiments, upon the user selecting the confirmation link 317, password GUI 320 is outputted to the display (see FIG. 3D), wherein password GUI 320 comprises a message informing the user that instructions for resetting the password have been sent to the email address provided if it is associated with the existing cloud-based user account. In some embodiments, password GUI 320 can comprise a Resend Email link 321 which, upon being selected, is configured to resend the instructions to the email address provided on password GUI 315 (FIG. 3C). According to an aspect of the embodiments, if the user selects the Resend Email link 321 a predetermined maximum number of times, then a modal (not illustrated) can be outputted which (1) informs the user that they have reached the limit to resend instructions along with a request that the user tries again after a predetermined time period has elapsed (e.g., after 24-hours), and (2) indicates to the user that the user should check trash/junk mail if the instructions are still not found.

In some embodiments, and turning to FIG. 3E, if the user inputs incorrect account credentials on the sign-in interface 325, a modal 326 can be outputted on the sign-in interface 325 which indicates to the user that a problem was encountered with the email address and password combination along with a message indicating the number of sign-in attempts remaining (e.g., “You have 2 sign-in attempts remaining”). According to an aspect of the embodiments, the user makes a maximum predetermined number of incorrect attempts to input account credentials, then a lock-out modal (not illustrated) is provided on the sign-in interface which indicates to the user that a problem was encountered with the email address and password combination along with a message indicating that no sign-in attempts remain (e.g., “You have 0 sign-in attempts remaining”). In some embodiments, though not illustrated, the lock-out modal can further inform the user that the user's cloud-based user account will be locked for a predetermined duration of time (e.g., five minutes, 10 minutes, 15 minutes, 30 minutes, one hour), and can comprise a confirmation button. According to an aspect of the embodiments, upon the user being locked-out of the user's cloud-based user account for the predetermined duration of time, a lock-out banner notification 330 (FIG. 3F) can be displayed on the sign-in interface 325, wherein the lock-out banner notification can comprise a message informing the user that the user's cloud-based user account has been locked due to too many failed sign-in attempts and that the user can try logging on again after the predetermined duration of time has elapsed (e.g., after five minutes).

According to an aspect of the embodiments, and upon the user selecting the Create Account button 312 start GUI 310 (FIG. 3B), an account creation interface is outputted (not illustrated) which comprises a plurality of account information fields through which the user can input a first name, a last name, and a date of birth that should be associated with the desired cloud-based user account. If the user inputs a date of birth that indicates the user is a minor (e.g., under the age of 18), then a series of minor account creation interfaces can be outputted so as to allow registration of the minor's cloud-based user account.

Specifically, and with reference to FIGS. 3G to 3I, block diagrams depicting example embodiments of minor account creation interfaces, or features related thereto, for use with the analyte monitoring software application are depicted. FIG. 3G depicts an account creation GUI 335 which comprises a message 336 comprising instructions related to setting up the minor's account (e.g., “Since the person who will be wearing the Sensor is a minor, a parent/legal guardian needs to create this account”). In some embodiments, account creation GUI 335 further comprises a warning informing the user that the date of birth is used to set up the cloud-based user account and is not used to determine whether the App is appropriate for use. The warning can further suggest that the user consult the App's user manual before utilizing the App. As depicted in FIG. 3G, account creation GUI 335 can further comprise a selectable continue button which, upon being selected outputs account creation GUI 340, as depicted in FIG. 3H.

With particular reference to FIG. 3H, account creation GUI 340 can be utilized to register the minor and create a minor cloud-based user account. Account creation GUI 340 can comprise one or more of the following: (1) a parent or legal guardian first name add field 341, which allows the parent or legal guardian to enter a first name; (2) a parent or legal guardian last name add field 342, which allows the parent or legal guardian to enter a last name; (3) a minor first name add field 343, which allows the parent or legal guardian to enter the minor's first name; (4) a minor last name add field 344, which allows the parent or legal guardian to enter the minor's last name; and (5) a minor date of birth field 345, which allows the parent or legal guardian to enter the minor's date of birth. In some embodiments, the parent or legal guardian first name add field 341 and the parent or legal guardian last name add field 342 are required fields that must be completed in order to create the minor's cloud-based user account. In some embodiments, the required parent or legal guardian first name add field 341 and the required parent or legal guardian last name add field 342 include an indicator which is configured to indicate that they are required entries. According to another aspect of the embodiments, account creation GUI 340 further comprises a next button 346 which is configured to be selectable upon all the required add fields on the account creation GUI 340 being completed. In some embodiments, the next button 346 comprises a colored portion to indicate that it is selectable. Further, upon the user selecting the next button 346, account creation GUI 350 is outputted, as depicted in FIG. 3I.

Specifically, and as shown in FIG. 3I, account creation GUI 350 comprises one or more of the following: (1) a parent or legal guardian email address add field 351; (2) a password add field 352; (3) and a confirm password add field 353 which allows the parent or legal guardian to confirm the password entered in password add field 352. Account creation GUI 350 can further comprise a selectable next button 354.

In some embodiments, if the email address entered in the parent or legal guardian email address add field 351 on account creation GUI 350 (FIG. 3I) is already associated with another cloud-based user account, then a modal (not illustrated) can be outputted upon the user selecting the next button 354, wherein the modal indicates that the email address is already associated with another cloud-based user account. Though not illustrated, the modal can further indicate that the user, or parent or legal guardian, should try signing into the already existing cloud-based user account, or use a different email address to create the minor's cloud-based user account. In some embodiments, the modal can comprise a Sign In link and cancel link.

In some embodiments, upon the user selecting the next button 354 on account creating GUI 350 (FIG. 3I), a HIPAA consent GUI can be displayed which prompts the user to either decline or allow authorization to use and disclose the user's health and demographic information.

Settings and/or tutorials interfaces, as will be described in further detail below, can be outputted subsequent to the HIPAA consent GUI being outputted.

Example Embodiments of Settings and Tutorial GUIs and Features Related thereto

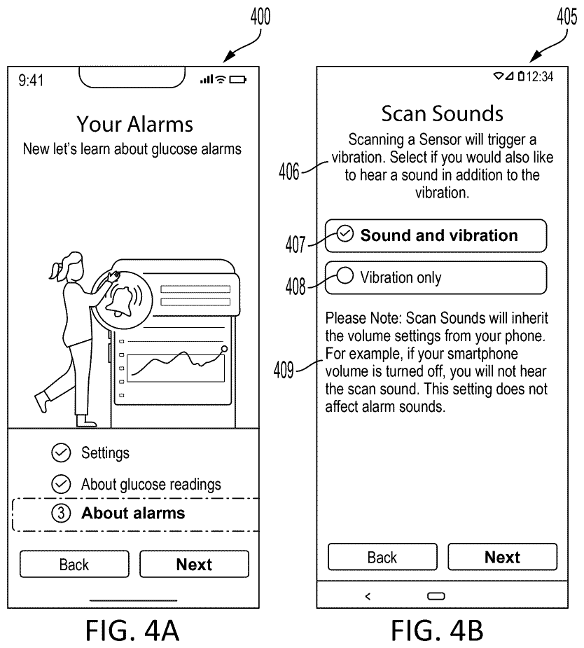

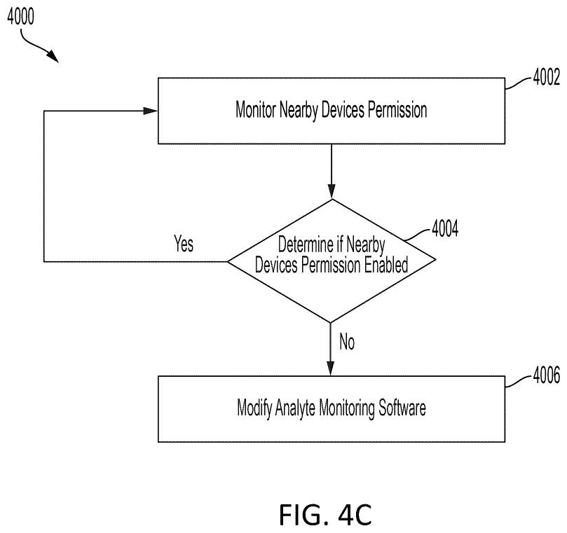

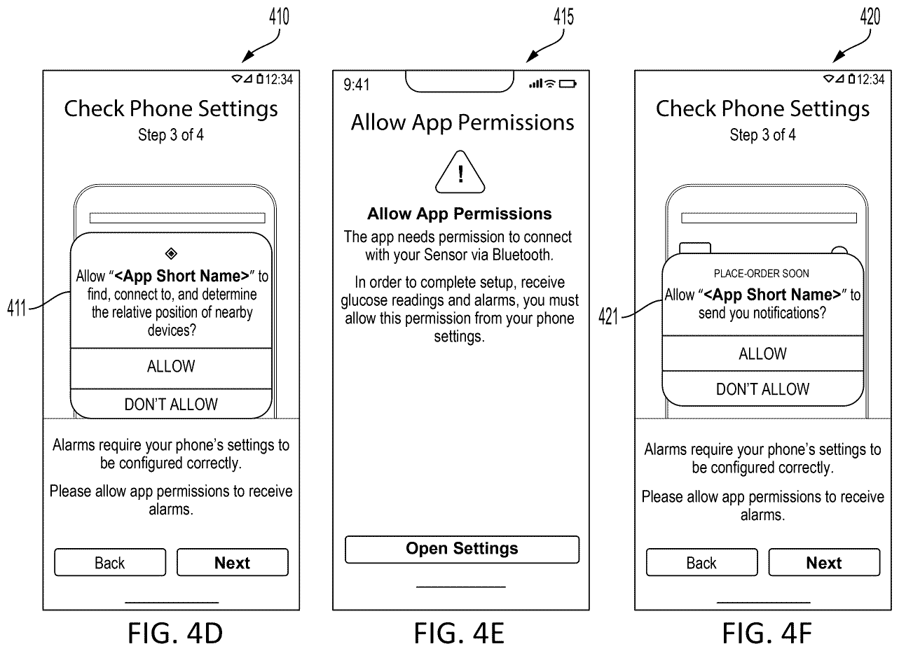

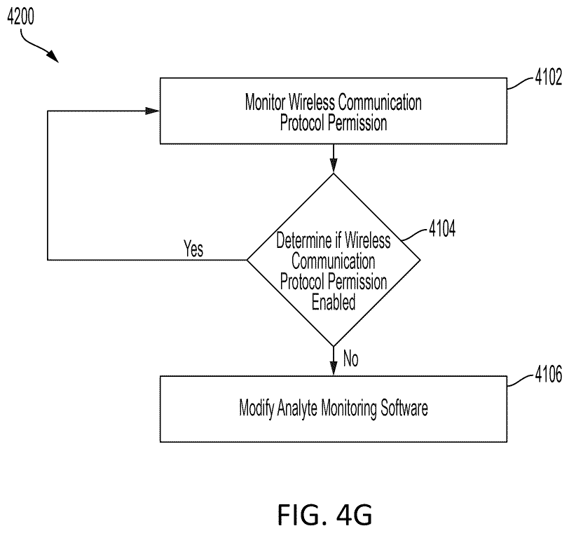

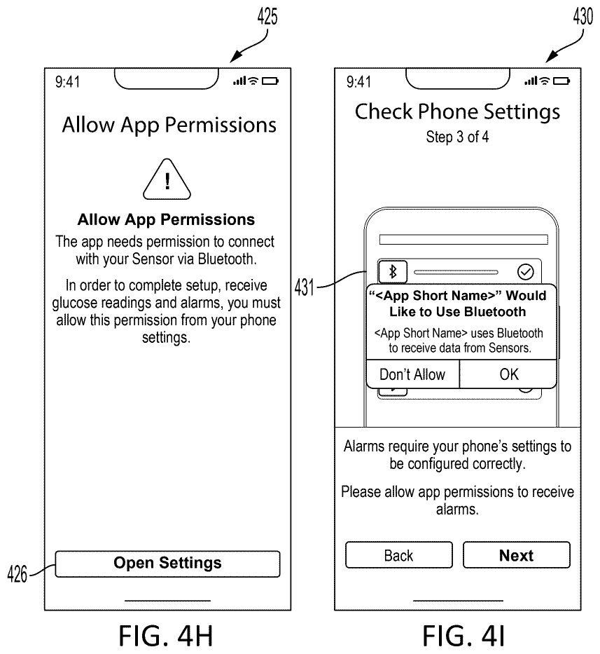

FIGS. 4A through 4I include block diagrams of example embodiments of tutorial and setting interfaces, or features related thereto, any of which can be utilized with the embodiments described herein. Referring to FIG. 4A, a tutorial GUI 400 is depicted. Specifically, tutorial GUI 400 can provide a brief explanation of the content which appears in the subsequent tutorial GUIs. For example, in some embodiments, tutorial GUI 400 can provide a brief explanation indicating that the subsequent tutorial GUIs will provide information relating to settings, glucose readings, and/or alarms. Specifically, in some embodiments, if alarms are enabled on the analyte monitoring software application or if at least one alarm is defaulted on, the tutorial GUI 400 can provide a brief explanation indicating that the subsequent tutorial GUIs will provide information relating to alarms. In some embodiments, tutorial GUI 400 can comprise a selectable Safety Information link which, upon being selected, outputs an interface comprising safety information related to the selected sensor type (e.g., a first sensor type or second sensor type). In some embodiments, tutorial GUI 400 can comprise a selectable back button. Further, tutorial GUI 400 can comprise a selectable next button. Following this, tutorial interfaces can be utilized to provide the user with a further introduction to the analyte monitoring software application and features related thereto.

For example, in some embodiments, and as depicted in FIG. 4B, a tutorial interface 405 can be displayed which allows the user to configure sensor scan sound settings. As shown in FIG. 4B, tutorial interface 405 can comprise one or more of the following: (1) a message 406 informing the user that scanning a sensor will trigger a vibration alone and further indicating that the user can select whether they would like to hear an auditory output in addition to the vibratory output; (2) a selectable sound and vibration option 407; (3) a selectable vibration option 408; (4) a notice 409 providing details related to the scan sound settings and its impact on alarm sounds (e.g., “Please Note: Scan Sounds will inherit the volume settings from your phone. For example, if your smartphone volume is turned off, you will not hear the scan sound. This setting does not affect alarm sounds.”). In some embodiments, tutorial interface 405 can also include a selectable back button and a selectable next button.

Turning next to FIG. 4C, an example embodiment of a method 4000 for monitoring a nearby devices permission and modifying an analyte monitoring software application in response to determining that the nearby devices permission is disabled. In some embodiments, an operating system of a reader device may include a nearby devices permission that determines whether an application installed on the reader device can find, connect to, and/or determine the position of other nearby devices. More specifically, granting an application with the nearby devices permission allows the application to search for nearby devices and networks using wireless communication protocols (e.g., Wi-Fi) that are separate from broader location services and/or navigational systems (e.g., GPS). Referring to FIG. 4C, at Step 4002, a nearby devices permission is monitored by the analyte monitoring software application. In some embodiments, this can be a periodic routine performed by the analyte monitoring software application while it is running (e.g., every 5 minutes, every 10 minutes, every 15 minutes, etc.). In some embodiments, this can be a routine performed by the analyte monitoring software application when it is first started or, in the alternative, in response to a predetermined event (e.g., viewing an alarms settings interface, foregrounding the App) or set of predetermined events (e.g., navigating from one GUI to another). In some embodiments, a nearby devices permission is monitored, requested, and required by the analyte monitoring software application during the onboarding process of the analyte monitoring software application.

At Step 4004, during the monitoring of the nearby devices permission, a determination is made as to whether the nearby devices permission is enabled for the analyte monitoring software application. If the permission is enabled, then the analyte monitoring software application continues to operate. If the nearby devices permission is disabled then, at Step 4006, the analyte monitoring software (e.g., the analyte monitoring software application) is modified in response thereto. In some embodiments, for example, the modification can be a blocking of the analyte monitoring software application such that no function or feature of the analyte monitoring software application is made available to the user until the nearby devices permission is enabled. In some embodiments, the modification can comprise an indication by the analyte monitoring software application that glucose readings and alarms are unavailable. In some embodiments, the modification can comprise an indication by the analyte monitoring software application that nearby device permissions are necessary in order to complete setup of the analyte monitoring software application. Further details regarding nearby device permission methods, GUIs, and features related thereto, are described in Appendix A, which is hereby incorporated by reference in its entirety for all purposes.

FIG. 4D is an example embodiment of a GUI 410 for an analyte monitoring software application, wherein GUI 410 includes a modal 411 for prompting the user to either allow or disallow the nearby devices permission with respect to the analyte monitoring software application. As described earlier, in some embodiments, the nearby devices permission allows the application to find, connect to, and determine the relative position of nearby devices. In this regard, according to many embodiments, selection of the “allow” option enables the nearby devices permission of the operating system with respect to the analyte monitoring software application.

Referring next to FIG. 4E, an example embodiment of an informational blocked app GUI 415 for an analyte monitoring software application is depicted. According to some embodiments, upon determination that the nearby devices permission is not enabled, the analyte monitoring software application can be blocked in response thereto. In some embodiments, for example, an informational blocked app GUI 415 can be presented in response to the determination that the nearby devices permission is not enabled, wherein the informational blocked app GUI 415 can indicate to the user that the analyte monitoring software application needs permission to connect with the analyte sensor via Bluetooth. Further operation of the analyte monitoring software application is prevented until the user enables the nearby devices permission. In some embodiments, the informational blocked app GUI 415 can further include a button 416 that, when selected, navigates the user to another GUI configured to allow the user to enable the nearby devices permission, such as a settings GUI that is part of the operating system of the reader device.

Further, FIG. 4F is an example embodiment of a GUI 420 for an analyte monitoring software application, wherein GUI 420 includes a modal 421 for prompting the user to select an option to either allow or disallow the analyte monitoring software application from sending notifications. According to many embodiments, the notifications setting is another permission granted by the operating system of the reader device that is separate from the nearby devices permission. In this regard, according to many embodiments, selection of the “allow” option enables a notifications permission of the operating system to allow the analyte monitoring software application to send notifications to the user.

Turning next to FIG. 4G, an example embodiment of a method 4100 for monitoring a wireless communication protocol permission (e.g., BLE permission) and modifying an analyte monitoring software application in response to determining that the wireless communication protocol permission is disabled. In some embodiments, an operating system of a reader device may include BLE permission that determines whether an application installed on the reader device can discover, connect to, and/or wirelessly communicate via BLE with other devices. Referring to FIG. 4G, at Step 4102, a BLE permission is monitored by the analyte monitoring software application. In some embodiments, this can be a periodic routine performed by the analyte monitoring software application while it is running (e.g., every 5 minutes, every 10 minutes, every 15 minutes, etc.). In some embodiments, this can be a routine performed by the analyte monitoring software application when it is first started or, in the alternative, in response to a predetermined event (e.g., viewing an alarms settings interface, foregrounding the App) or set of predetermined events (e.g., navigating from one GUI to another). In some embodiments, a BLE permission is monitored, requested, and required by the analyte monitoring software application during the onboarding process of the analyte monitoring software application.

At Step 4104, during the monitoring of the BLE permission, a determination is made as to whether the BLE permission is enabled for the analyte monitoring software application. If the permission is enabled, then the analyte monitoring software application continues to operate. If the BLE permission is disabled then, at Step 4106, the analyte monitoring software (e.g., the analyte monitoring software application) is modified in response thereto. In some embodiments, for example, the modification can be a blocking of the analyte monitoring software application such that no function or feature of the analyte monitoring software application is made available to the user until the BLE is enabled. In some embodiments, the modification can comprise an indication by the analyte monitoring software application that glucose readings and alarms are unavailable. In some embodiments, the modification can comprise an indication by the analyte monitoring software application that BLE permissions are necessary in order to complete setup of the analyte monitoring software application.

FIG. 4H is an example embodiment of an informational blocked app GUI 425 for an analyte monitoring software application is depicted. According to some embodiments, upon determination that the BLE permission is not enabled, the analyte monitoring software application can be blocked in response thereto. In some embodiments, for example, an informational blocked app GUI 425 can be presented in response to the determination that the BLE permission is not enabled, wherein the informational blocked app GUI 425 can indicate to the user that the analyte monitoring software application needs permission to connect with the analyte sensor via Bluetooth. Further operation of the analyte monitoring software application is prevented until the user enables the BLE permission. In some embodiments, the informational blocked app GUI 425 can further include a button 426 that, when selected, navigates the user to another GUI configured to allow the user to enable the BLE permission, such as a settings GUI that is part of the operating system of the reader device.

Further, FIG. 4I, is an example embodiment of a GUI 430 for an analyte monitoring software application, wherein GUI 430 includes a modal 431 for prompting the user to select an option to either allow or disallow the analyte monitoring software application from using Bluetooth. As described earlier, in some embodiments, the BLE permission allows the application to discover, connect to, and/or communicate with other devices. In this regard, according to many embodiments, selection of the “allow” option enables the BLE permission of the operating system with respect to the analyte monitoring software application. In some embodiments, GUI 430 comprising modal 431 is presented only once by the analyte monitoring software application.

Further details regarding account and settings GUIs and features related thereto are described in U.S. Publ. No. 2022/0248988, which is incorporated by reference in its entirety for all purposes.

Example Embodiments of Sensor Activation and Results GUIs and Features Related thereto

Various example embodiments relating to sensor activation and sensor results interfaces, or methods and/or features related thereto, for an analyte monitoring software application of an analyte monitoring system will now be described.

According to an aspect of the embodiments, the analyte monitoring software application is configured to pair with a plurality of different types of sensors (also herein referred to as “sensor types”). In some embodiments, the sensor type can be an analyte sensor, such as a first type of glucose sensor or a second type of glucose sensor, wherein the first type of glucose sensor is different from the second type of glucose sensor. In some embodiments, the sensor type can be a glucose sensor, a glucose-ketone sensor, lactate, alcohol, or any other analyte sensor (or combination of analytes). For example, the first type of sensor can be a sensor configured to measure glucose levels, and the second type of sensor can be a sensor configured to measure both glucose and ketone levels, or vice versa. In some examples, the first sensor type and the sensor type may sense the same analyte(s) but the first sensor and second sensor types may correspond to different sensor versions/iterations that contain different sensor hardware, firmware, and/or features (e.g., different communications protocols). Further, according to some embodiments, the first type of sensor (or first sensor type) can be a component of a sensor control device that can be configured to transmit analyte data on demand, and the second type of sensor (or second sensor type) is configured to operate with a sensor control device that transmits analyte data via streaming protocol, or vice versa. In some embodiments, the first type of sensor is manufactured by a first manufacturer and the second type of sensor is manufactured by a second manufacturer, wherein the first manufacturer is different from the second manufacturer.

In some embodiments, the analyte monitoring software application is configured to pair with the first type of sensor and the second type of sensor, wherein the first type of sensor is different than the second type of sensor. Those of skill in the art will appreciate that various other sensor types can be utilized and paired with the analyte monitoring software application described herein without departing from the scope of the present disclosure. Those of skill in the art will also appreciate that the first type of sensor and/or second type of sensor can be configured to measure various other types of analytes/analyte levels without departing from the scope of the present disclosure. Additionally, those of skill in the art will appreciate that the analyte monitoring software application can be configured to pair with more than two types of sensors without departing from the scope of the present disclosure.

According to some embodiments, and depending on which sensor type is paired with the analyte monitoring application, the analyte monitoring application can modify its functionality and can be configured to output different GUIs, data, and information depending on the sensor type paired therewith. In some embodiments, the analyte monitoring software application can only pair with one sensor at a time. In this manner if the analyte monitoring software application activates one sensor, then it cannot activate a second sensor without discontinuing interaction with the first sensor. According to an aspect of the embodiments, a new sensor can be detected when it is activated, e.g., by establishing a wireless communication link between a new sensor control device and the reader device. In some embodiments, a new sensor can be activated during an NFC scanning process. In some embodiments, the analyte monitoring software application can activate a new sensor only after the user allows the required permissions (e.g., BLE permissions and nearby devices permissions). According to some embodiments, upon sensor data being received from an activated sensor, glucose information, trend information, historical information, and alarms relating thereto can be viewed on the analyte monitoring software application. In some embodiments, the analyte monitoring software application is further configured to interact and communicate with supported cloud-based applications.

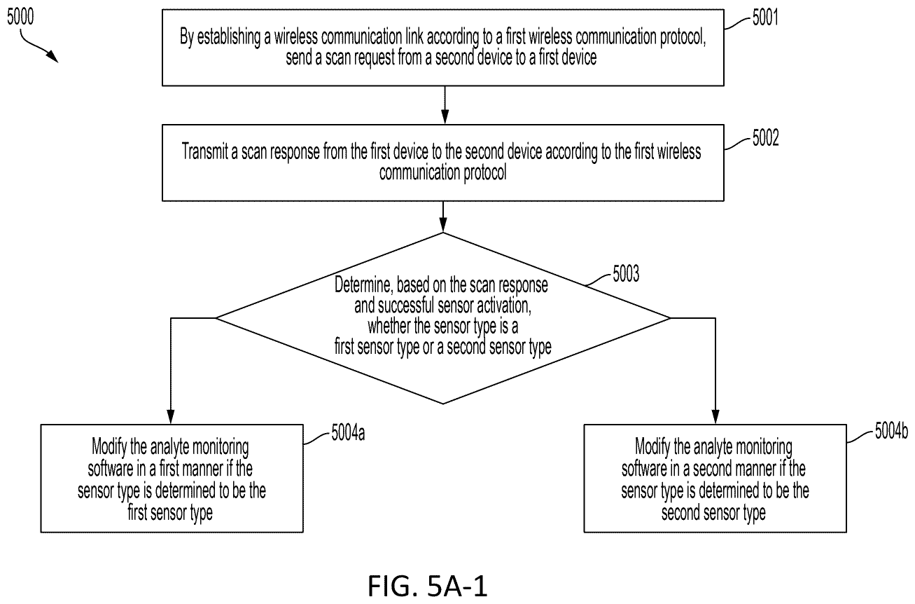

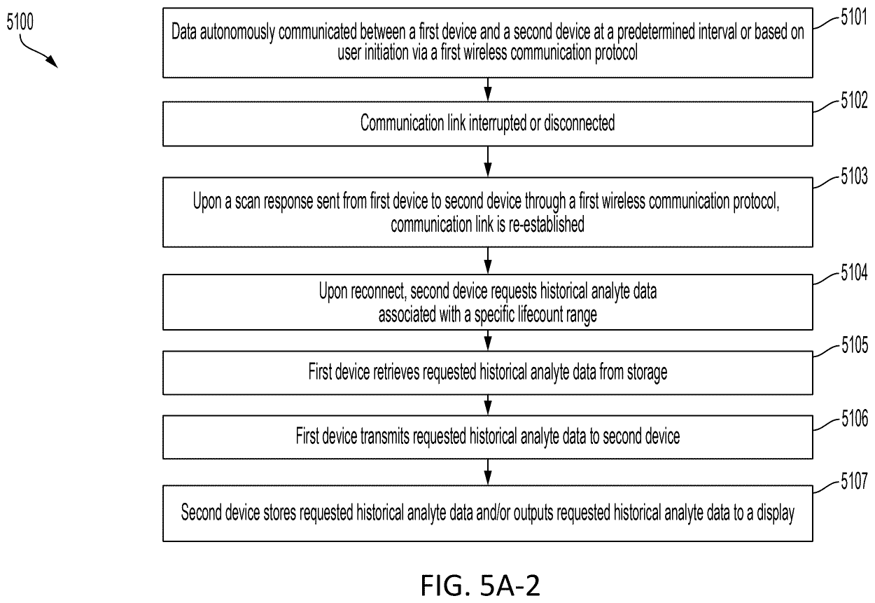

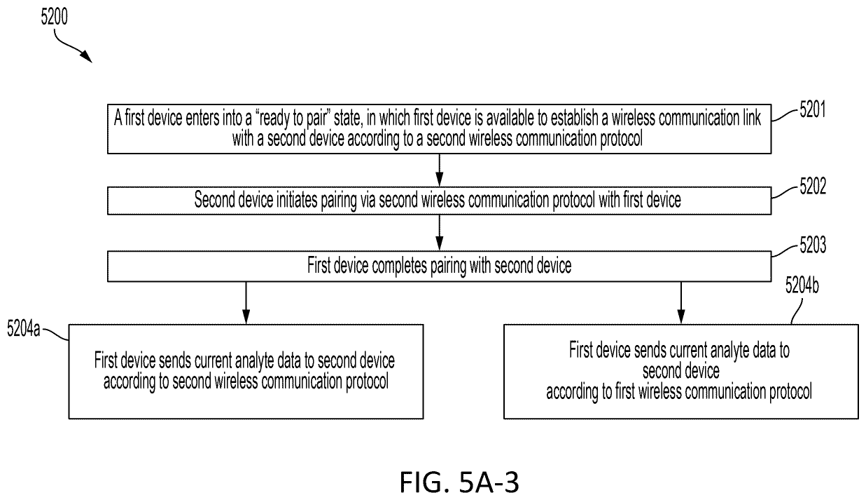

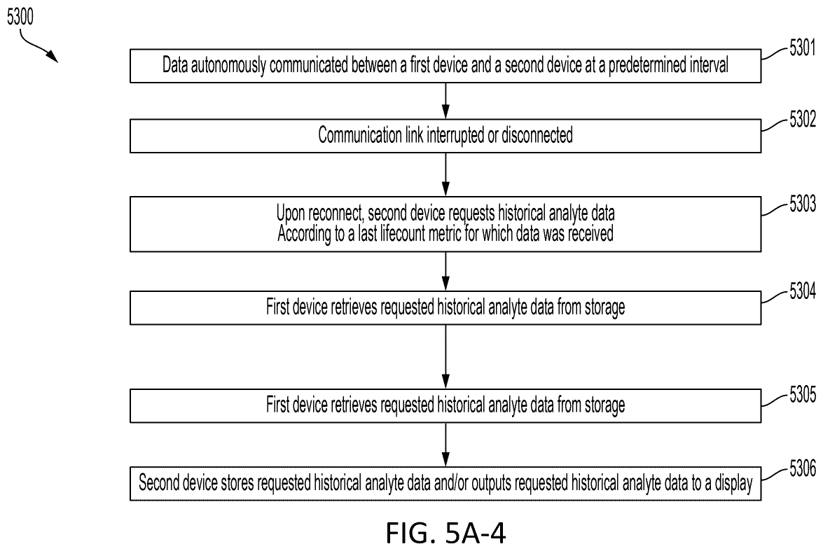

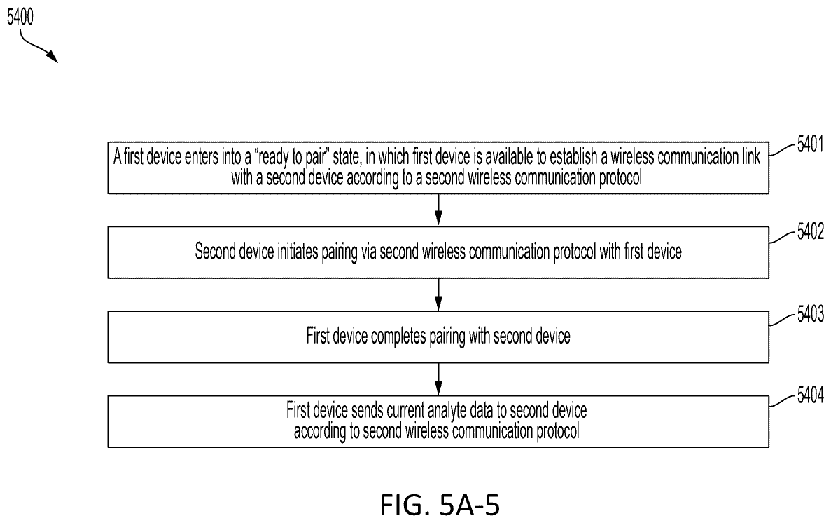

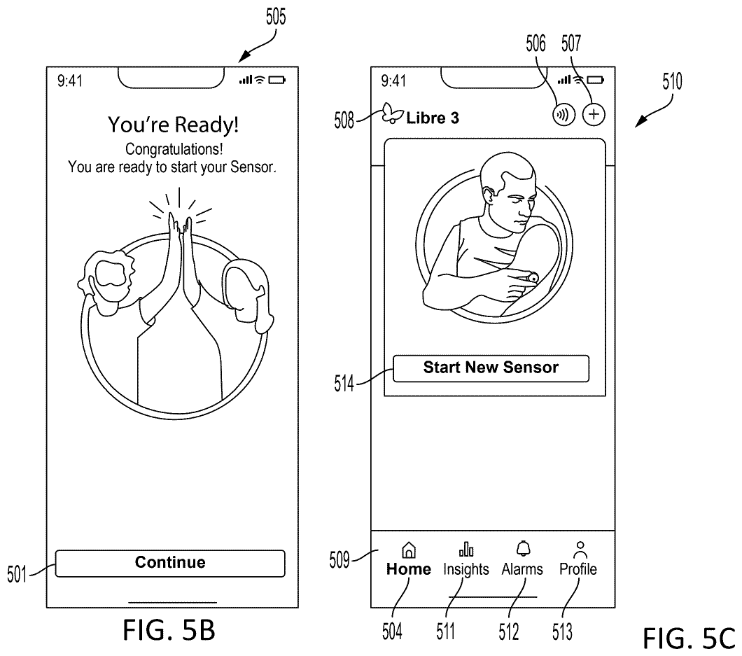

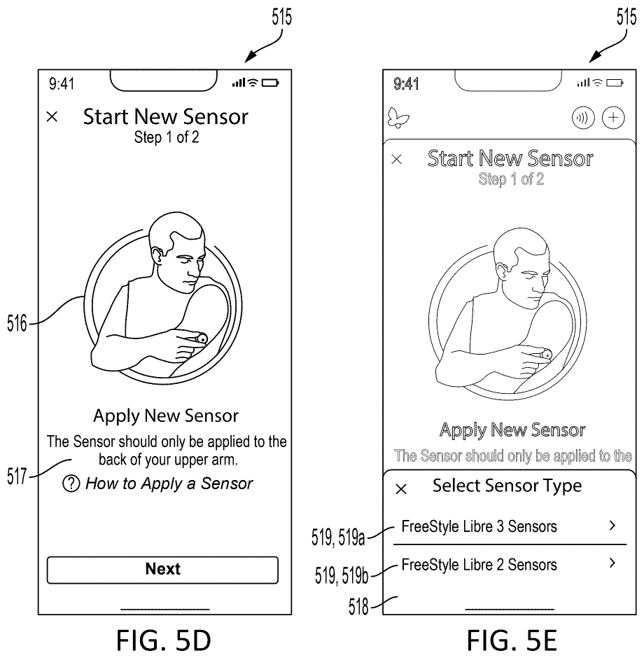

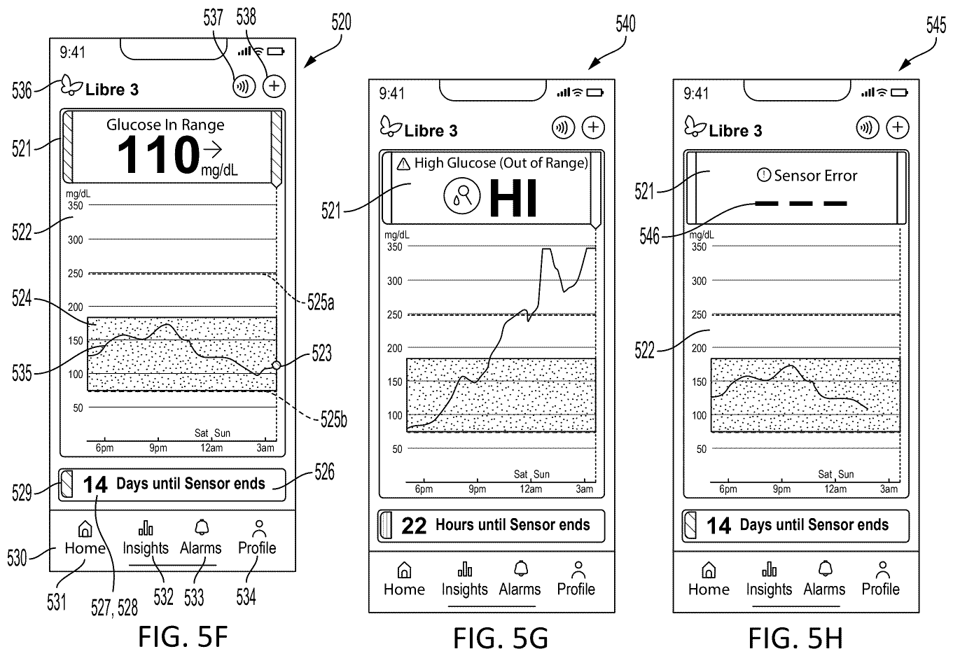

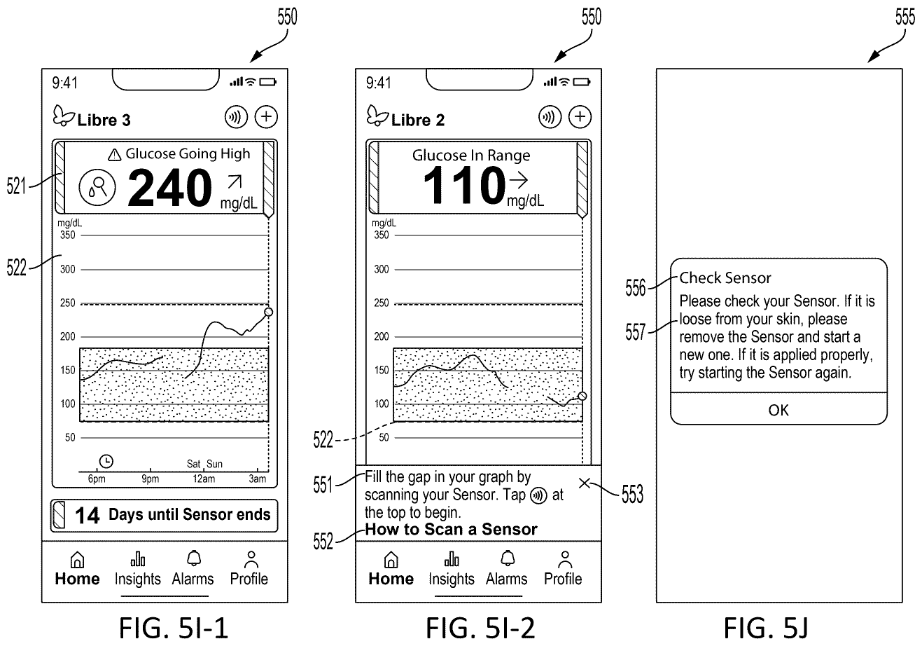

FIG. 5A-1 depicts a flow diagram of an example embodiment of a method 5000 for pairing a sensor with the analyte monitoring software application. Referring to FIG. 5A-1, at Step 5001, by establishing a wireless communication link according to a first wireless communication protocol (e.g., an NFC protocol) between a first device (e.g., a sensor control device comprising the sensor) and a second device (e.g., a reader device) comprising an installed analyte monitoring software application, sending a scan request from the second device to the first device comprising the sensor through the first wireless protocol. At Step 5002, causing a transmission of a scan response from the first device to the second device comprising the installed analyte monitoring software application according to the first wireless communication protocol. At step 5003, determining, based on the scan response and successful activation of the sensor, whether the sensor type is a first sensor type or a second sensor type. If the sensor type is the first sensor type, then at Step 5004a, modifying the analyte monitoring software (e.g., the analyte monitoring software application) in a first configuration in response thereto. In some embodiments, for example, the first configuration can comprise the analyte monitoring software application (1) outputting GUIs comprising a first sensor type indicator, (2) outputting a series of interfaces (e.g., tutorial interfaces) and features related thereto corresponding to the first sensor type, (3) requiring a first method of sensor data backfilling, (4) and/or utilizing a first method of receiving, via a communication protocol, sensor data (also herein referred to as “analyte data,” “glucose data,” “ketone data,” “alcohol data,” “lactate data,” “data indicative of an analyte level” or “data indicative of a glucose level”) for the graphical display of analyte results/data. Those of skill in the art will appreciate that the analyte monitoring software application can be modified in various other ways depending on the determined sensor type without departing from the scope of the present disclosure. In some embodiments, upon successfully scanning a sensor, the reader device will output a vibratory, or vibratory and audible output so as to indicate a successful scan.

Specifically, in some embodiments, and as will be described in further detail below, if it is determined through successful sensor activation that the sensor type is the first sensor type, then interfaces subsequently outputted can comprise a first sensor type indicator configured to indicate the type of sensor paired with the analyte monitoring software application (e.g., by providing a textual description of a brand name associated with the first sensor type). In some embodiments, the first sensor type indicator can be positioned in an upper-right corner (or any other area) of the interfaces for the analyte monitoring software application. Further, in some exemplar embodiments, if it is determined that the sensor type is the first sensor type and in the event of a disconnection event or condition (e.g., interruption to a wireless communication link through, for example, a gap in the Bluetooth connectivity), the user must scan the first type of sensor, utilizing the first wireless communication protocol (e.g., NFC protocol), in order to backfill up historical analyte data or other information associated with a specific lifecount range (e.g., last eight hours of sensor analyte data and other information). Those of skill in the art will recognize that a lifecount can be a value associated with the operating life of a sensor control device or a sensor. For example, according to some embodiments, the lifecount can be a numerical value that is incremented a predetermined amount at a predetermined frequency during the operating life of a sensor control device or a sensor. In some embodiments, for example, the lifecount can be incremented by “one” every second, such as a counter. In other embodiments, the lifecount can be a timestamp. According to another aspect of some embodiments, the incrementing of the lifecount value can be initiated when the sensor control device is activated. In other embodiments, the incrementing of the lifecount value can be initiated when the sensor is inserted in the user's body. Those of skill in the art will appreciate that the initiation of the incrementing of the lifecount value can be associated with other events (e.g., upon pairing with the analyte monitoring software application, upon user initiation via the analyte monitoring software application, etc.). Further details regarding the lifecount value and features relating thereto are described in U.S. Publ. Nos. 2022/0263905 and 2019/0369083, which are incorporated by reference in their entireties for all purposes.

According to another aspect of the embodiments, if the sensor type is determined to be the second sensor type upon successful sensor activation at step 5003 (FIG. 5A-1), then at Step 5004b, the analyte monitoring software application is modified in a second configuration in response thereto. In some embodiments, for example, the second configuration can comprise the analyte monitoring software application (1) outputting GUIs comprising a second sensor type indicator, different than the first sensor type indicator (2) outputting a series of interfaces (e.g., tutorial interfaces) and features related thereto corresponding to the second sensor type (3) requiring a second method of sensor data backfilling, (4) and/or utilizing a second method of receiving, via a communication protocol, sensor data for the graphical display of analyte results/data. Those of skill in the art will appreciate that the analyte monitoring software application can be modified in various other ways depending on the determined sensor type without departing from the scope of the present disclosure.

Specifically, in some embodiments, and as will be described in further detail below, if it is determined that the sensor type is the second sensor type, then interfaces subsequently outputted can comprise a second sensor type indicator configured to indicate the type of sensor paired with the analyte monitoring software application (e.g., by providing a textual description of a brand name associated with the second sensor type). In some embodiments, the second sensor type indicator can be positioned in an upper-right corner (or any other area) of the interfaces for the analyte monitoring software application. Further, in some exemplar embodiments, if it is determined that the sensor type is the second sensor type and in the event of a disconnection event or condition (e.g., interruption to a wireless communication link through, for example, a gap in the Bluetooth connectivity), the full duration of any data gap can be configured to automatically be backfilled upon a BLE reconnection.

Specifically, FIG. 5A-2 depicts a flow diagram depicting an example embodiment of a method 5100 for data backfilling in an analyte monitoring system comprising the first sensor type. According to one aspect of the embodiments, method 5100 can be implemented to provide data backfilling between a sensor control device 102 comprising the first type of sensor and a reader device 120. At Step 5101, analyte data and other information is autonomously communicated between a first device and a second device at a predetermined interval or, communicated based on user initiation through the first wireless communication protocol. In some embodiments, the first device can be a sensor control device 102 comprising the first type of sensor, and the second device can be a reader device 120 comprising the installed analyte monitoring software application. According to one aspect of the embodiments, analyte data and other information can include, but is not limited to, one or more of: data indicative of an analyte level in a bodily fluid, a rate-of-change of an analyte level, a predicted analyte level, a low or a high analyte level alert condition, a sensor fault condition, or a communication link event. According to another aspect of the embodiments, autonomous communications at a predetermined interval can comprise streaming analyte data and other information according to a standard wireless communication network protocol, such as a Bluetooth or Bluetooth Low Energy protocol, at one or more predetermined rates (e.g., every minute, every five minutes, every fifteen minutes, etc.). In some embodiments, different types of analyte data or other information can be autonomously communicated between the first and second devices at different predetermined rates (e.g., historical glucose data every 5 minutes, current glucose value every minute, etc.).

At Step 5102, a disconnection event or condition occurs that causes an interruption to the communication link between the first device and the second device. As described above, the disconnection event can result from the second device (e.g., reader device 120, smart phone, etc.) running out of battery power or being powered off manually by a user. A disconnection event can also result from the first device being moved outside a wireless communication range of the second device, from the presence of a physical barrier that obstructs the first device and/or the second device, or from anything that otherwise prevents wireless communications from occurring between the first and second devices.

At Step 5103, the communication link is re-established between the first device and the second device upon the first device sending a scan response to the second device through a first wireless communication protocol (e.g., by utilizing an NFC protocol, the user scans the sensor control device comprising the first type of sensor with the reader device. At Step 5104, upon reconnection, the second device can send a request to the first device for historical analyte data associated with a specific lifecount range (e.g., up to last eight hours of historical sensor data)

At Step 5105, upon receiving the request, the first device retrieves the requested historical analyte data from storage (e.g., non-transitory memory of sensor control device 102), and subsequently transmits the requested historical analyte data to the second device at Step 5106 At Step 5107, upon receiving the requested historical analyte data, the second device stores the requested historical analyte data in storage (e.g., non-transitory memory of reader device 120). According to one aspect of the embodiments, when the requested historical analyte data is stored by the second device, it can be stored along with the associated lifecount metric. In some embodiments, the second device can also output the requested historical analyte data to a display of the second device, such as, for example to a glucose trend graph of a sensor results or home GUIs, such as those described with respect to FIGS. 5F to 5I-2. For example, in some embodiments, the requested historical analyte data can be used to fill in gaps in a glucose trend graph by displaying the requested historical analyte data along with previously received analyte data.

Furthermore, those of skill in the art will appreciate that the method of data backfilling can be implemented between multiple and various devices in an analyte monitoring system, wherein the devices are in wired or wireless communication with each other.

Additionally, in some embodiments, and as will be described in further detail below, the analyte monitoring software application can provide an in-app notification as well as a lock screen notification to the user as a reminder to scan for backfill of the data once per sensor wear when a disconnection event or condition has been detects that causes an interruption to the communication link between the first device comprising the first type of sensor and the second device (e.g., data gap of a predetermined duration has been detected, such as a data gap of more than one hour, two hours, three hours).

Further, according to another aspect of the embodiments, if it is determined step 5003 (FIG. 5A-1) that the sensor type is the first sensor type, the second device initiates a pairing sequence via a wireless communication link according to a second wireless communication protocol (e.g., Bluetooth or Bluetooth Low Energy). Specifically, FIG. 5A-3 depicts a flow diagram depicting an example embodiment of a method 5200 for pairing the first device comprising the first type of sensor with the second device comprising the installed analyte monitoring software application.

Specifically, at step 5201, the first device enters into a “ready to pair” state, in which the first device is available to establish a wireless communication link with second device according to the second wireless communication protocol. According to an aspect of the embodiments, the existing wireless communication link can comprise a link established according to a second wireless communication protocol that is different from the first wireless communication protocol. In some embodiments, for example, the second wireless communication protocol can be a Bluetooth or Bluetooth Low Energy protocol.

At Step 5202, the second device initiates a pairing sequence via the second wireless communication protocol (e.g., Bluetooth or Bluetooth Low Energy) with the first device. Subsequently, at Step 5203, first device completes the pairing sequence with second device. At Step 5204a, the first device can begin sending current glucose data to second device according to the second wireless communication protocol. In some embodiments, at Step 5204a, the first device can begin sending current analyte data (e.g., glucose data) to the second device according to the second wireless communication protocol (Bluetooth or Bluetooth Low Energy), and in a similar manner to the process in which the first device comprising a second type of sensor begins sending current analyte data to second device, as is described in further detail below with respect FIG. 5A-5 and step 5404.

In some embodiments, for example, current glucose data can be wirelessly transmitted to the second device at a predetermined interval (e.g., every minute, every two minutes, every five minutes). Alternatively or additionally, at step 5204b, the first device can begin sending current glucose data to second device according to the first wireless communication protocol (e.g., NFC protocol). For example, after a user-initiated scan, via an NFC protocol, the sensor control device can begin sending current glucose data to reader device. As such, analyte data and other information graphically displayed on, e.g., the home interfaces described herein are automatically refreshed and streamed (1) at a predetermined frequency (e.g., every 30-seconds, every minute, every five minutes, every five minutes, every fifteen minutes) based on the sensor data received via the second wireless communication protocol (e.g., BLE communication) and/or (2) according to the first wireless communication protocol, e.g., after a user-initiated scan via an NFC protocol.

According to another aspect of the embodiments, if the sensor type is determined to be the second sensor type at step 5003 (FIG. 5A-1), then a second method of backfilling can be utilized in the event of a disconnection event or condition.

Turning to FIG. 5A-4, flow diagram depicting an example embodiment of a method 5300 for data backfilling in an analyte monitoring system comprising the second sensor type is depicted. According to one aspect of the embodiments, method 5300 can be implemented to provide data backfilling between a sensor control device 102 comprising the first type of sensor and a reader device 120. At Step 5301, analyte data and other information is autonomously communicated between a first device and a second device at a predetermined interval. In some embodiments, the first device can be a sensor control device 102 comprising the first type of sensor, and the second device can be a reader device 120 comprising the installed analyte monitoring software application. According to one aspect of the embodiments, analyte data and other information can include, but is not limited to, one or more of: data indicative of an analyte level in a bodily fluid, a rate-of-change of an analyte level, a predicted analyte level, a low or a high analyte level alert condition, a sensor fault condition, or a communication link event. According to another aspect of the embodiments, autonomous communications at a predetermined interval can comprise streaming analyte data and other information according to a standard wireless communication network protocol, such as a Bluetooth or Bluetooth Low Energy protocol, at one or more predetermined rates (e.g., every minute, every five minutes, every fifteen minutes, etc.). In some embodiments, different types of analyte data or other information can be autonomously communicated between the first and second devices at different predetermined rates (e.g., historical glucose data every 5 minutes, current glucose value every minute, etc.).

At Step 5302, a disconnection event or condition occurs that causes an interruption to the communication link between the first device and the second device. As described above, the disconnection event can result from the second device (e.g., reader device 120, smart phone, etc.) running out of battery power or being powered off manually by a user. A disconnection event can also result from the first device being moved outside a wireless communication range of the second device, from the presence of a physical barrier that obstructs the first device and/or the second device, or from anything that otherwise prevents wireless communications from occurring between the first and second devices.

At Step 5303, the communication link is re-established between the first device and the second device (e.g., the first device comes back into the wireless communication range of the second device). Upon reconnection, the second device requests historical analyte data according to a last lifecount metric for which data was received. As previously described, according to one aspect of some embodiments, the lifecount metric can be a numeric value that is incremented and tracked on the second device in units of time (e.g., minutes), and is indicative of an amount of time elapsed since the sensor control device was activated. For example, in some embodiments, after the second device (e.g., reader device 120, smart phone, etc.) re-establishes a Bluetooth wireless communication link with the first device (e.g., sensor control device 120), the second device can determine the last lifecount metric for which data was received. Then, according to some embodiments, the second device can send to the first device a request for historical analyte data and other information having a lifecount metric greater than the determined last lifecount metric for which data was received.

In some embodiments, the second device can send a request to the first device for historical analyte data or other information associated with a specific lifecount range, instead of requesting historical analyte data associated with a lifecount metric greater than a determined last lifecount metric for which data was received.

At Step 5304, upon receiving the request, the first device retrieves the requested historical analyte data from storage (e.g., non-transitory memory of sensor control device 102), and subsequently transmits the requested historical analyte data to the second device at Step 5305. At Step 5306, upon receiving the requested historical analyte data, the second device stores the requested historical analyte data in storage (e.g., non-transitory memory of reader device 120). According to one aspect of the embodiments, when the requested historical analyte data is stored by the second device, it can be stored along with the associated lifecount metric. In some embodiments, the second device can also output the requested historical analyte data to a display of the second device, such as, for example to a glucose trend graph of a sensor results or home GUI, such as those described with respect to FIGS. 5F to I-2. For example, in some embodiments, the requested historical analyte data can be used to fill in gaps in a glucose trend graph by displaying the requested historical analyte data along with previously received analyte data.

Furthermore, those of skill in the art will appreciate that the method of data backfilling can be implemented between multiple and various devices in an analyte monitoring system, wherein the devices are in wired or wireless communication with each other.

Moreover, according to another aspect of the embodiments, if it is determined step 5003 (FIG. 5A-1) that the sensor type is the second sensor type, the second device initiates a pairing sequence via a wireless communication link according to a second wireless communication protocol (e.g., Bluetooth or Bluetooth Low Energy). Specifically, FIG. 5A-5 depicts a flow diagram depicting an example embodiment of a method 5400 for pairing the first device comprising the second type of sensor with the second device comprising the installed analyte monitoring software application.

Specifically, at step 5401, the first device enters into a “ready to pair” state, in which the first device is available to establish a wireless communication link with second device according to the second wireless communication protocol. According to an aspect of the embodiments, the existing wireless communication link can comprise a link established according to a second wireless communication protocol that is different from the first wireless communication protocol. In some embodiments, for example, the second wireless communication protocol can be a Bluetooth or Bluetooth Low Energy protocol.

At Step 5402, the second device initiates a pairing sequence via the second wireless communication protocol (e.g., Bluetooth or Bluetooth Low Energy) with the first device. Subsequently, at Step 5403, first device completes the pairing sequence with second device. At Step 5404, the first device can begin sending current analyte data (e.g., glucose data) to second device according to the second wireless communication protocol. In some embodiments, for example, current analyte data can be wirelessly transmitted to the second device at a predetermined interval (e.g., every minute, every two minutes, every five minutes). As such, analyte data and other information graphically displayed on, e.g., the home interfaces described herein are automatically refreshed and streamed at a predetermined frequency (e.g., every 30-seconds, every minute, every five minutes, every five minutes, every fifteen minutes) based on the sensor data received via the second wireless communication protocol (e.g., BLE communication).

According to yet another aspect of the embodiments, if the user transitions between a plurality of activated sensor (e.g., from a first activated sensor to a second activated sensor), wherein the plurality of activated sensors are different types (e.g., wherein the first activated sensor is a different sensor type than the second activated sensor, or when the first activated sensor is the first type of sensor and the second activated sensor is the second type of sensor, or vice versa), then the analyte monitoring software application is further configured to display historical analyte data from the first activated sensor, and historical and current analyte data from the second activated sensor so as to output GUIs comprising analyte data merged from all sensors that have been or are paired with the analyte monitoring software application. In this manner, data and information outputted through the interfaces of the analyte monitoring software application (e.g., interfaces related to sensor results, trend graphs, reports, logbooks, etc.), comprises merged, contiguous data received from more than one sensor control device/sensor.

Further details regarding sensor activation, pairing, data communication, and backfilling are described in U.S. Publ. Nos. 2022/0248988 and 2021/0378601, which are hereby incorporated by reference in their entirety for all purposes.

FIGS. 5B to 5H-2 are block diagrams depicting example embodiments of sensor activation interfaces, and sensor results or home interfaces, or features related thereto, for an analyte monitoring software application, any of which can be utilized with the embodiments described herein. Turning to FIG. 5B, a GUI 505 is depicted, wherein GUI 505 comprises a message congratulating the user and indicating that the user is ready to start a sensor. In some embodiments, GUI 500 comprises a selectable continue button 501 which upon being selected, can output a GUI through which the user can begin the setup to pair a new sensor.

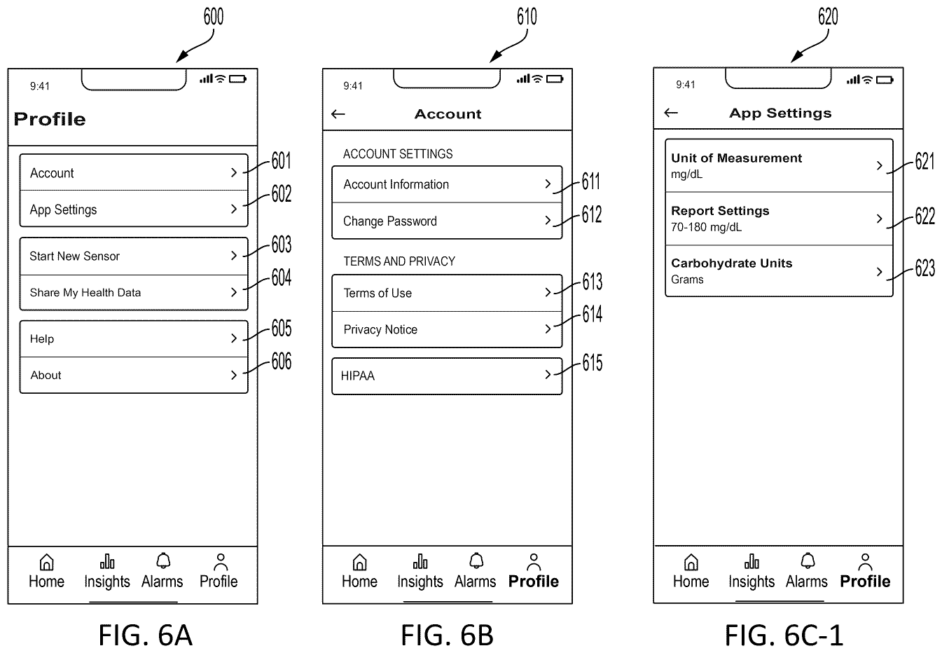

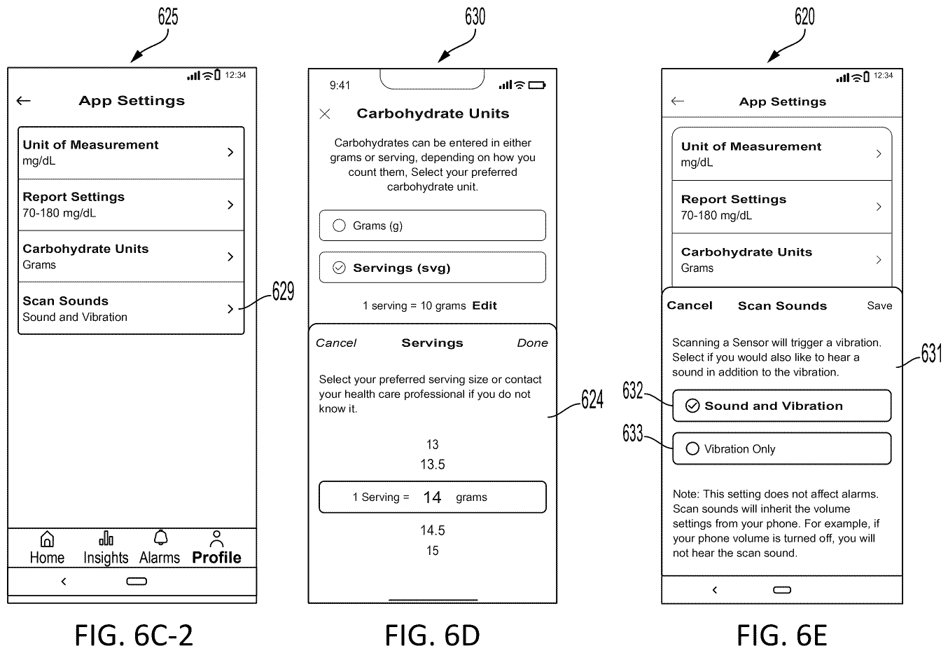

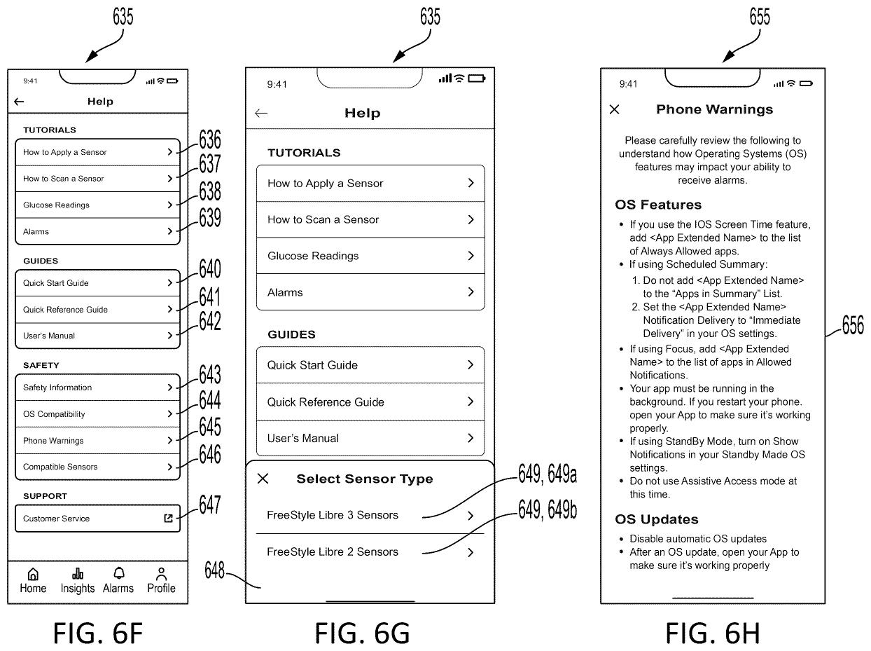

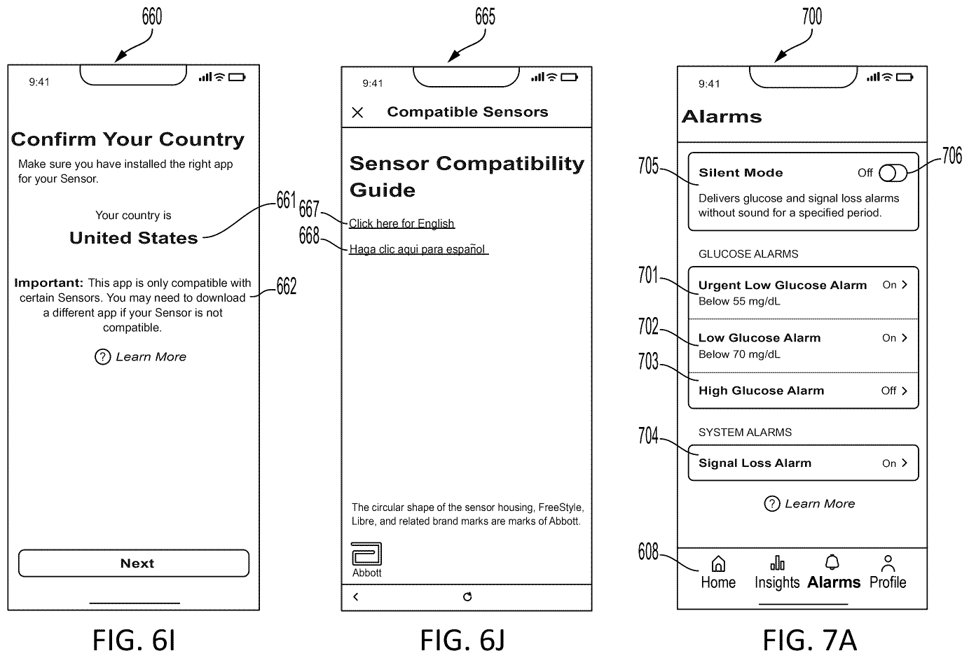

In some embodiments, a profile interface (described in further detail below with reference to FIG. 6A) is outputted comprising a selectable start new sensor section which, upon being selected, allows the user to begin the setup process of pairing a new sensor.

Further, referring to FIG. 5C, a block diagram of an example embodiment of a home GUI 510 is depicted. Specifically, in some embodiments, home GUI 510 can comprise one or more of the following: (1) a scan button 506 which, upon being selected, allows the user to initiate scanning of a new sensor; (2) a selectable “+” button 507; (3) a sensor type indicator 508 (e.g., a first sensor type indicator 508, such as a textual description of a brand name associated with the first sensor type, or a second sensor type indicator 508, such as a textual description of a brand name associated with the second sensor type) configured to indicate the sensor type upon successful activation thereof; and (4) a bottom bar navigation menu 509. According to an aspect of the embodiments, home GUI 510 and the other home interfaces described herein are configured to output historical and current analyte data, and sensor results, when the analyte monitoring software application is paired with either a first type of sensor or a second type of sensor. In some embodiments, the home GUIs described herein are configured to update at a predetermined frequency (e.g., every thirty seconds, every minute, every five minutes, etc.) so as to display real-time, or near real-time, current analyte data.

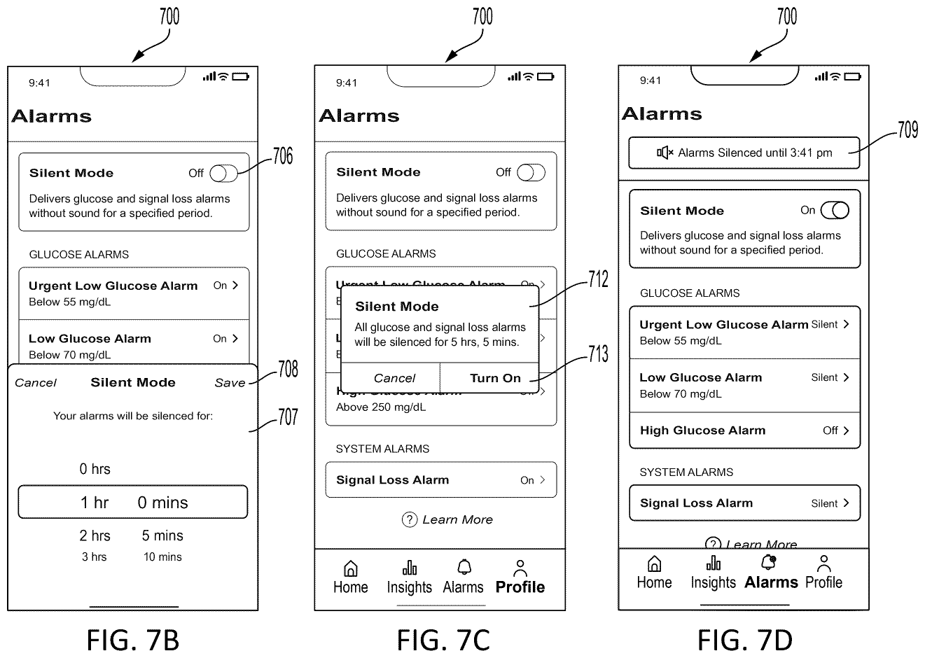

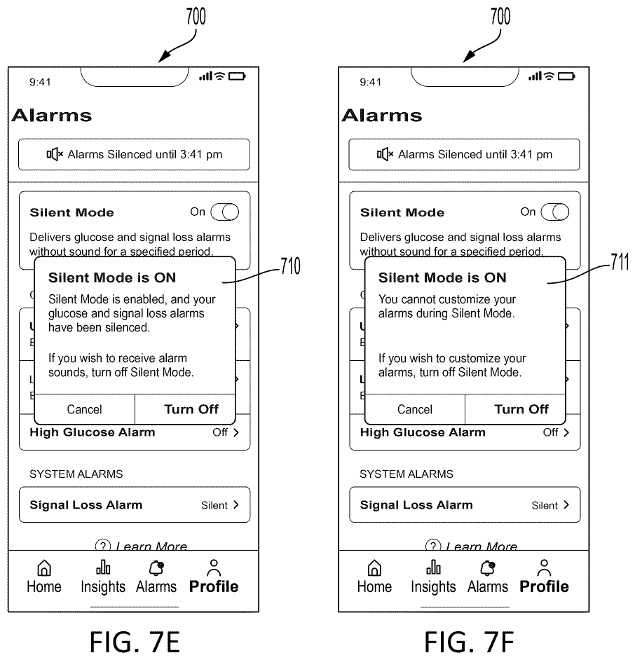

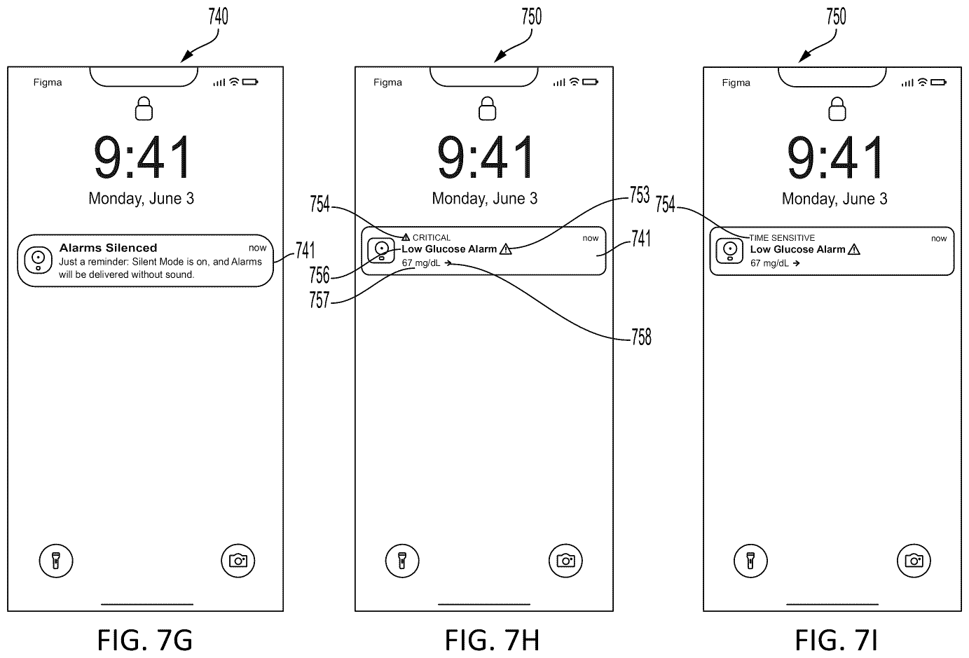

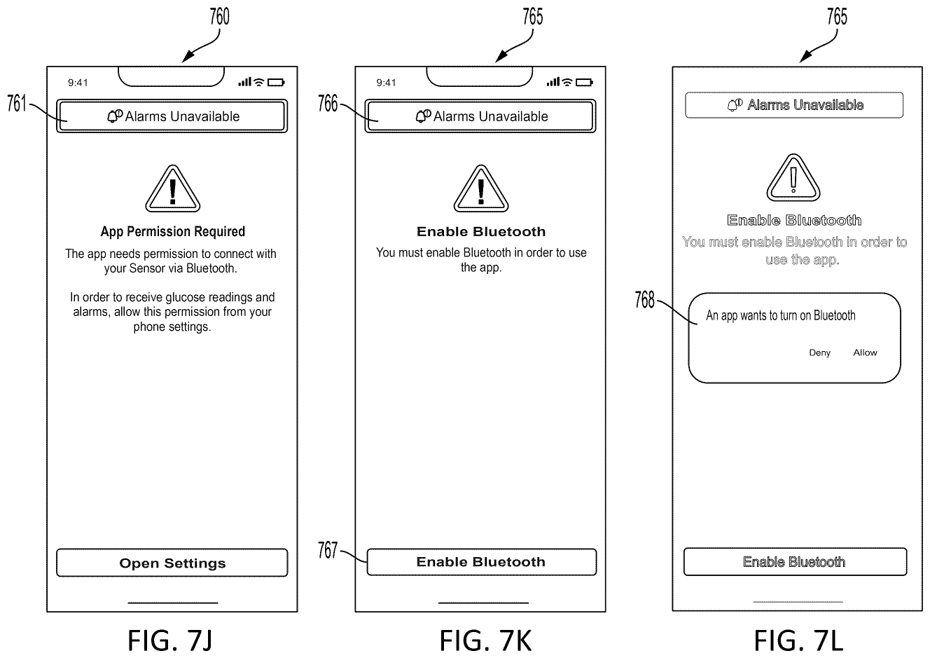

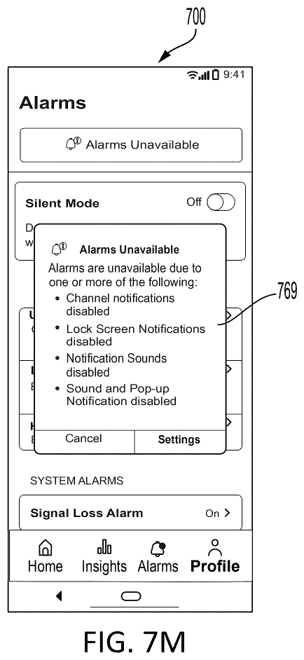

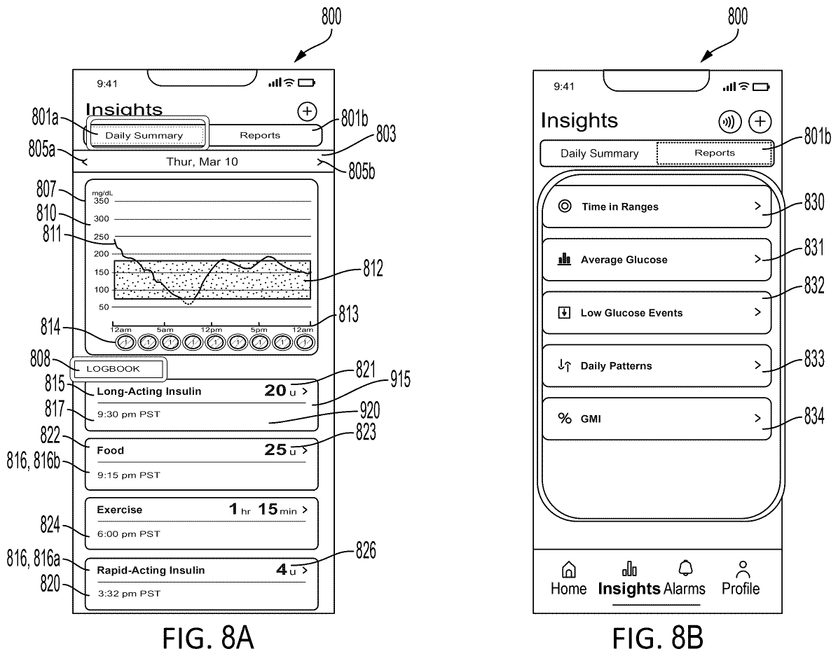

In some embodiments, the bottom bar navigation menu 509 comprises a plurality of selectable icons. In some embodiments (though not illustrated), the bottom bar navigation menu 509 comprises three selectable icons. In other embodiments, and as depicted in FIG. 5C, the bottom bar navigation menu 509 comprises four selectable icons: (1) a selectable home icon 504 which, upon being selected, outputs one of the home interfaces described herein (e.g., home GUIs depicted in FIGS. 5C, and 5F to 5I-2); (2) a selectable insights icon 511 which, upon being selected, outputs interfaces comprising insights, reports, logbooks, daily summary data, including but not limited to daily graph (e.g., outputs insights GUI 800, as shown in FIG. 8A or 8B); (3) a selectable alarms icon 512 which, upon being selected, outputs interfaces relating to alarms (e.g., alarm settings GUI 700, as shown in FIG. 7A); and (4) a selectable profile icon 513 which, upon being selected, outputs an interface related to the user's profile or account (e.g., profile GUI 600, as shown in FIG. 6A). Those of skill in the art will appreciate that the bottom bar navigation menu and features related thereto that are described herein with respect to home GUI 510 are meant to be illustrative only, and that the individual elements, or any combination of elements, depicted and/or described for a particular embodiment or figure are freely combinable with any other element, or any combination of other elements, depicted and/or described with respect to any of the other embodiments (e.g., interface embodiments described herein).

With reference to FIG. 5C, in instances in which a new sensor has not yet been activated, home GUI 510 can further comprise a selectable Start New Sensor button 514 which outputs a GUI that allows the user to scan or start a new sensor. In some embodiments, upon the user selecting the Start New Sensor button 514, a series of interfaces can be presented to assist the user in scanning/starting a new sensor.

Specifically, and with reference to FIG. 5D, a sensor application GUI 515 is depicted, wherein sensor application GUI 515 is presented to assist the user in scanning/starting a new sensor. Sensor application GUI 515 can display a reminder that the sensor should only be applied to the back of the user's arm as a message associated with a graphic 516, as seen in FIG. 5D. The sensor application GUI 515 can further include a link 517 to instructions regarding how to apply the sensor. Specifically, upon the user selecting link 517, a bottom sheet 518 (FIG. 5E) is displayed on sensor application GUI 515, wherein the bottoms sheet 518 comprises a plurality of selectable sensor type options 519). More specifically, and as depicted in FIG. 5E, in some embodiments, the plurality of selectable sensor type options 519 includes a selectable sensor type option corresponding to a first sensor type 519a (e.g., (e.g., a textual description of a brand name associated with the first sensor type) and a selectable sensor type option corresponding to a second sensor type 519b (e.g., a textual description of a brand name associated with the second sensor type). Even more specifically, in some embodiments, if the user selects the sensor type option corresponding to the first sensor type 519a, a series of tutorial interfaces corresponding to the first sensor type can be presented to assist the user in activating and/or applying the first sensor type. In like manner, in some embodiments, if the user selects the sensor type option corresponding to the second sensor type 519b, a series of tutorial interfaces corresponding to the second sensor type can be presented to assist the user in activating and/or applying the second sensor type. According to some embodiments, the series of tutorial interfaces corresponding to the first sensor type are different from the series of tutorial interfaces corresponding to the second sensor type. Those of skill in the art will appreciate that various other selectable sensor type options 519 corresponding to various other sensor types and instructions related thereto can be displayed on bottom sheet 518 without departing from the scope of the present disclosure.

Further, in some embodiments, though not illustrated, once the user has started a new sensor and if the new sensor is in a warm-up period (e.g., a first 60-minutes after activating a new sensor), a modal can be outputted which indicates to the user that the sensor has been started but is not ready to be used yet.

FIGS. 5F through 5I-2 depict additional example embodiments of home GUIs (also herein referred to as sensor results GUIs) for an analyte monitoring software application. According to some embodiments, the home GUIs described herein can also be displayed during a sensor warmup period. The home GUIs described herein can also be outputted in response to the user selecting the home icon on the bottom bar navigation menu on any of the interfaces described herein. In some embodiments, the home GUIs described herein can reflect data indicative of the glucose level and data corresponding to the first sensor type if it is determined that the activated glucose sensor is the first sensor type. Further, in some embodiments, the home GUIs described herein can reflect data indicative of the glucose level and data corresponding to the second sensor type if it is determined that the glucose sensor is the second sensor type.

Referring first to FIG. 5F, home GUI 520 depicts an interface comprising a first portion 521 that can include a numeric representation of a current analyte concentration value (e.g., a current glucose value), a directional arrow to indicate an analyte trend direction, and a text description to provide contextual information such as, for example, whether the user's analyte level is in range (e.g., “Glucose in Range”). According to some embodiments, the text description can provide contextual information to indicate whether user's analyte level is above or below range (e.g., “Low Glucose” or “High Glucose”). According to some embodiments, the text description can provide contextual information to indicate whether an out-of-range condition is present (i.e., the user's current analyte level is either above or below a predetermined reportable analyte level range). Further, according to some embodiments, for example, the text description can provide contextual information to indicate trend information (e.g., “Glucose Going High” or “Glucose Going Low”). In some embodiments, the first portion 521 can also comprise a check blood glucose icon. In some embodiments, the check blood glucose icon is displayed for a predetermined period of time following sensor activation (e.g., for the first 12-hours after sensor activation).

First portion 521 can also comprise a color or shade that is indicative of an analyte concentration or trend. For example, as shown in FIG. 5F, first portion 521 is a green shade, indicating that the user's analyte level is within a target range. According to some embodiments, for example, a red shade can indicate an analyte level below a low analyte level threshold, an orange shade can indicate an analyte level above a high analyte level threshold, and a yellow shade can indicate an analyte level outside a target range. In addition, according to some embodiments, home GUI 520 also includes a second portion 522 comprising a graphical representation of analyte data.

In particular, second portion 522 includes an analyte trend graph reflecting an analyte concentration, as shown by the y-axis, over a predetermined time period, as shown by the x-axis. In some embodiments, the predetermined time period can be shown in three-hour increments with one-hour increment tick marks, with a total of twelve hours of data. Those of skill in the art will appreciate, however, that other time increments and durations of analyte data can be utilized and are fully within the scope of the present disclosure. The analyte trend graph can also include an analyte trend line 535 which can reflect historical analyte levels over time and a current analyte data point 523 to indicate the current analyte concentration value (shown in green to indicate that the current value is within the target range). According to another aspect of many embodiments, the home GUI 510 is configured to be interactive. In some embodiments, for example, when the user interacts with a point on the analyte trend line 535 (e.g., by touching a point on the analyte trend line 535), the first portion 521 and second portion 522 can be updated to display corresponding first historical analyte data (if any) and corresponding information associated with the point of the analyte trend line 535 being interacted with by the user. Second portion 522 can also include a shaded green area 524 to indicate a target analyte range, and two dotted lines 525a and 525b to indicate, respectively, a first alarm threshold (e.g., high analyte alarm threshold, such as a high glucose alarm threshold) and a second alarm threshold (e.g., a low analyte alarm threshold, such as a low glucose alarm threshold) if alarms are enabled, wherein each alarm threshold corresponds to a specific alarm.

According to some embodiments, home GUI 520 can also include a third portion 526 comprising a graphical indicator 527 and textual information representative of a remaining amount of sensor life. According to an aspect of the embodiments, the graphical indicator 527 can include a numerical value 528 corresponding to the number of days, hours, or minutes remaining in the lifetime of the sensor (e.g., “14”) along with a tab 529 comprising a colored portion indicative of a status of the remaining lifetime of the sensor. For example, in some embodiments, if the remaining lifetime of the sensor exceeds one day, then the tab 529 can include a green colored portion (see, e.g., FIGS. 5F, 5H, and 5I-1 to 5I-2). Further, in some embodiments, if the remaining lifetime of the sensor is less than one day (e.g., if the remaining lifetime of the sensor is calculated in hours, minutes, or seconds), then the tab 529 can include a red colored portion (see, e.g., FIG. 5G). Further, the textual information can indicate the units of measure (e.g., days, hours, minutes, or seconds) used to calculate the remaining lifetime of the sensor (e.g., “Days until Sensor ends,” “Hours until Sensor ends,” “Minutes until Sensor ends,” or “Seconds until Sensor ends”). For example, if the remaining lifetime of the sensor is 13 days, then the numerical value 528 (e.g., “13”) and the textual information (e.g., “Days until Sensor ends”), collectively, indicate that there is “13 Days until Sensor ends.” Those of skill in the art will recognize that various other graphical indications, colors and textual messages can be utilized to visually illustrate the remaining lifetime of the sensor without departing from the scope of the present disclosure.

According to another aspect of some embodiments, and with reference to FIG. 5G, home GUI 520 can also include a sensor type indicator 536 (e.g., a first sensor type indicator 536, such as, a textual description of a brand name associated with the first sensor type, or a second sensor type indicator 536, such as a textual description of a brand name associated with the second sensor type) configured to indicate the sensor type currently paired with the analyte monitoring software application. Home GUI 520 can further comprise a scan button 537 which, upon being selected, allows the user to initiate scanning of a new sensor, and a selectable “+” button 538.

Still with reference to FIG. 5F, home GUI 520 can also include a bottom bar navigation menu 530 comprising a plurality of selectable icons: (1) a selectable home icon 531 which, upon being selected, outputs one of the home interfaces described herein (e.g., home GUIs depicted in FIGS. 5C, and 5F to 5I-2); (2) a selectable insights icon 532 which, upon being selected, outputs interfaces comprising insights, reports, logbooks, daily summary data, including but not limited to daily graph (e.g., outputs insights GUI 800, as shown in FIG. 8A or 8B); (3) a selectable alarms icon 533 which, upon being selected, outputs interfaces relating to alarms (e.g., alarm settings GUI 700, as shown in FIG. 7A); and (4) a selectable profile icon 534 which, upon being selected,, outputs an interface related to the user's profile or account (e.g., alarm settings GUI 700, as shown in FIG. 7A).

Referring next to FIG. 5G, another example embodiment of a home GUI 540 is depicted. According to the depicted embodiments, home GUI 540 includes first portion 521 which is also shown in an orange shade to indicate that the user's analyte levels are above a high glucose threshold. As can be seen in FIG. 5G, first portion 521 does not report a numeric value but instead displays the text “HI” to indicate that the current analyte concentration value is outside a glucose reporting range high limit, or that an out-of-range condition is present. Although not depicted in FIG. 5G, those of skill in the art will understand that, conversely, an analyte concentration below a glucose reporting range low limit will cause first portion 521 not to display a numeric value, but instead, the text “LO”.

FIG. 5H is another example embodiment of a home GUI 545. According to the depicted embodiments, home GUI 545 depicts first portion 521 when there is a sensor error. According to one aspect of the embodiments, first portion 521 includes three dashed lines 546 in place of the current analyte concentration value to indicate that a current analyte value is not available. In some embodiments, three dashed lines 546 can indicate one or more error conditions such as, for example, (1) a no signal condition; (2) a signal loss condition; (3) sensor too hot/cold condition; or (4) a glucose level unavailable condition. Furthermore, as can be seen in FIG. 5H, first portion 521 comprises a gray shading (instead of green, yellow, orange, or red) to indicate that no current analyte data is available. In addition, according to another aspect of the embodiments, second portion 522 can be configured to display the historical analyte data in the analyte trend graph, even though there is an error condition preventing the display of a numeric value for a current analyte concentration in first portion 521. However, as shown in FIG. 5H, no current analyte concentration value data point is shown on the analyte trend graph of second portion 522.

FIG. 5I-1 is another example embodiment of a home GUI 550. According to the depicted embodiments, home GUI 550 includes first portion 521 which is shown in a yellow shade to indicate that the user's current analyte level is outside a target range. In addition, according to the depicted embodiments, first portion 521 of home GUI 550 includes the text, “GLUCOSE GOING HIGH,” which can indicate to the user that his or her analyte concentration value is predicted to rise above a predicted high analyte level threshold within a predetermined amount of time (e.g., predicted glucose will rise above 250 mg/dL within 15 minutes). Those of skill in the art will understand that if a user's analyte level is predicted to drop below above a predicted low analyte level threshold within a predetermined amount of time, home GUI 550 can display a “GLUCOSE GOING LOW” message.

Further, as shown in FIG. 5H-1, the analyte trend graph of the second portion 522 includes a gap in the displayed analyte data. Gaps in analyte data and other information can result from interruptions to communication links between various devices in an analyte monitoring system (e.g., a gap in the BLE connectivity). In this manner, data backfilling is required to fill the gap in the analyte trend graph.

In some embodiments, and as best depicted in FIG. 5I-2, home GUI 550 can further comprise a backfill callout 551. Specifically, the backfill callout 551 can comprise a message informing the user that he or she can fill the gap in the analyte trend graph by scanning the paired sensor. The message can also inform the user that the user can tap or select on scan button 528 at the top of home GUI 550 to begin scanning. Further, in some embodiments, the backfill callout 551 can include a link 552 which, upon being selected, outputs information on how to scan a sensor. In some embodiments, the backfill callout 551 includes a dismiss button 553 which, upon being selected, removes the backfill callout 551 from the display. In some embodiments, the backfill callout 551 covers or overlays the graphical indicator and textual information representative of a remaining amount of sensor life, and a portion of the second portion 522.

Specifically, in some embodiments, the backfill callout 551 is configured to be outputted only when one of the following occurs for the gap(s) in analyte data and/or other information resulting from interruptions to communication links between various devices in an analyte monitoring system: (1) when the interruption is greater than or equal to a duration of a predetermined amount of time (e.g., greater than or equal to 60 minutes, greater than or equal to 120 minutes), and is the first occurrence of an interruption during the current sensor wear duration; or (2) after whatever is the latest between up to eight hours duration or up to the latest real-time or current sensor data scan. According to some embodiments, the backfill callout 551 is configured to remain on the home GUI 550 unless dismissed by one of the following dismissal actions: (1) the user scans the sensor successfully; (2) end of gap in data occurred more than a predetermined number of hours ago (e.g., more than eight hours ago); (3) user dismisses backfill callout 511 by selecting the dismiss button; or (4) the sensor ends/terminates.

Turning to FIG. 5J, a GUI 555 comprising sensor modal 556 is depicted. Specifically, in some embodiments, sensor modal 556 is outputted when the sensor insertion detection has failed. According to some embodiments, sensor modal 556 comprises a message 567 relating to the failed insertion detection along with instructions on how to remedy the issue (e.g., Please check your sensor. If it is loose from your skin, please remove the Sensor and start a new one. If it is applied properly, try starting the Sensor again.”).

Further details regarding home GUIs (or sensor results GUIs), and features related thereto, are described U.S. Publ. Nos. 2022/0248988, 2021/0378601, 2019/0183393, and U.S. patent application Ser. No. 18/380,590, filed Oct. 16, 2023, all of which are hereby incorporated by reference in their entirety for all purposes.

Example Embodiments of Profile and Account GUIs and Features Related thereto

FIGS. 6A through 6J depict block diagrams of example embodiments of profile and account interfaces, or features related thereto, any of which can be utilized with the embodiments described herein.

FIG. 6A is a block diagram depicting an example embodiment of a profile GUI 600 for an analyte monitoring software application of an analyte monitoring system, any of which can be utilized with the embodiments described herein. According to some embodiments, profile GUI 600 can be outputted in response to the user selected the profile icon on the bottom bar navigation menu displayed on any of the interfaces described herein. In some embodiments, profile GUI 600 includes a plurality of selectable profile sections, including but not limited to, an account section 601, a settings section 601, a start new sensor section 603, a HIPAA section 604 (e.g., “Share my health data”), a help section 605, and an about section 606. In some embodiments, profile GUI 600 further comprises a bottom bar navigation menu comprising a selectable home icon which, upon being selected, outputs one of the home interfaces described herein (e.g., home GUIs depicted in, e.g., FIGS. 5C, and 5F to 5I-2); (2) a selectable insights icon which, upon being selected, outputs interfaces comprising reports, logbooks, daily summary data, including but not limited to daily graphs (e.g., outputs insights GUI 800, as shown in FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., profile GUI 600, as shown in FIG. 6A).

Specifically, upon the user selecting the account section 601, an account GUI 610 (FIG. 6B) is outputted which includes an account settings section and a notices section. As shown in FIG. 6B, in some embodiments, the account settings section comprises one or more plurality of selectable sections, including but not limited to: (1) an account information section 611 which, upon being selected, outputs an interface through which the user can input or update user-information associated with the cloud-based user account (e.g., first name, last name, date of birth, country of residence, and/or email address); and (2) a password section 612 which, upon being selected, outputs an interface through which the user can update or change the password associated with the cloud-based user account. In some embodiments, upon the user selecting the account information section 611 and inputting on an interface user-information determined to be for a minor (e.g., the date of birth indicates that the user is under the age of 18), a modal is outputted indicating to the user that the account cannot be managed by a minor and further requesting that the user create a new account if the person who is wearing the sensor is a minor.

Further, and still with reference to FIG. 6B, the notices section of account GUI 610 can comprise one or more plurality of selectable sections, including but not limited to: (1) a Terms of Use section 613 which, upon being selected outputs an interface comprising the Terms of Use for the analyte monitoring software application; (2) a Privacy Notice section 614 which, upon being selected outputs an interface comprising the Privacy Notice for the analyte monitoring software application; and (3) a HIPAA section 615 which, upon being selected outputs an interface comprising the HIPAA consent GUI for the analyte monitoring software application

In some embodiments, account GUI 610 (FIG. 6B) further comprises a bottom bar navigation menu comprising a selectable home icon which, upon being selected, outputs one of the home interfaces described herein (e.g., home GUIs depicted in, e.g., FIGS. 5C, and 5F to 5I-2); (2) a selectable insights icon which, upon being selected, outputs interfaces comprising reports, logbooks, daily summary data, including but not limited to daily graphs (e.g., outputs insights GUI 800, as shown in FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., profile GUI 600, as shown in FIG. 6A).

According to an aspect of the embodiments, upon the user selecting the settings section 602 on profile GUI 600 (FIG. 6A), a settings GUI 620 is outputted, as depicted in FIG. 6C-1. Settings GUI 620 can be presented through a mobile operating system (e.g., an iOS system), and can comprise one of more of the following selectable settings sections: (1) a unit of measurement settings section 621 which, upon being selected, outputs an interface which allows the user to review the glucose unit of measurement automatically set based on the user's country; (2) a report settings section 622 which, upon being selected, outputs a modal through which the user can configure the target analyte range settings associated with and displayed on analyte trend graphs of various interfaces described herein; and (3) a macronutrient units settings section 623 which, upon being selected, outputs interfaces which allow the user to update the macronutrient unit settings for the analyte monitoring application (e.g., carbohydrate units, such as grams or servings). Specifically, in some embodiments, a macronutrient unit configurator modal 624 (FIG. 6D) can be outputted to a GUI 630 which allows the user to edit or customize a preferred serving size associated with a macronutrient unit (e.g., one serving size equals 14 grams).

In some embodiments, settings GUI 620 (FIG. 6C-1) further comprises a bottom bar navigation menu comprising a selectable home icon which, upon being selected, outputs one of the home interfaces described herein (e.g., home GUIs depicted in FIGS. 5C, and 5F to 5I-2); (2) a selectable insights icon which, upon being selected, outputs interfaces comprising reports, logbooks, daily summary data, including but not limited to daily graphs (e.g., outputs insights GUI 800, as shown in FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., alarm settings GUI 700, as shown in FIG. 7A).

According to another aspect of the embodiments, upon the user selecting the settings section 602 on profile GUI 600 (FIG. 6A), a settings GUI 625 is outputted, as depicted in FIG. 6C-2. Settings GUI 625 can be presented through a different mobile operating system (e.g., an iOS system) than that used with respect to settings GUI 620 (FIG. 6C-1). Settings GUI 625 is similar to settings GUI 620 (FIG. 6C-1), except that it can further comprise a scan sounds settings section 629 which, upon being selected, outputs a scan sounds configurator halfsheet 631 (FIG. 6E) on settings GUI 620, wherein halfsheet 631 comprises a first scan sounds option 632 configured to set the scan sounds setting such that scanning a sensor can trigger an audible and vibratory alert (e.g., sound and vibration), and a second scan sounds option 633 configured to set the scan sound settings such that scanning a sensor can trigger only a vibratory alert (e.g., a vibration only).

According to another aspect of the embodiments, upon the user selecting the start new sensor section 603 on profile GUI 600 (FIG. 6A), the user can begin the setup process of pairing a new sensor. In some embodiments, upon the user selecting the start new sensor section 603 on profile GUI 600 (FIG. 6A), a sensor modal (not illustrated) is outputted to profile GUI 600 if a sensor is currently in use, wherein the sensor modal comprises a message indicating to the user that starting a new sensor will end the sensor currently being utilized. According to an aspect of the embodiments, the analyte monitoring software application can only receive glucose readings from a sensor that is activated. Further, the analyte monitoring software application cannot be used with a sensor that has been activated by another application or reader device. In some embodiments, though not illustrated, the sensor modal further comprises a query asking whether the user would like to start the new sensor now, along with a selectable deny button and a selectable confirmation button.

According to yet another aspect of the embodiments, upon the user selecting the HIPAA section 604 on profile GUI 600 (FIG. 6A), the HIPAA consent GUI can be displayed.

Further, according to some embodiments, upon the user selecting the help section 605 on profile GUI 600 (FIG. 6A), outputs a help GUI 635, as depicted in FIG. 6F. Specifically, and as shown in FIG. 6F, help GUI 635 can comprise a menu of options related to tutorials, guides, safety, and/or support information. In some embodiments, help GUI 635 (FIG. 6F) further a bottom bar navigation menu comprising a selectable home icon which, upon being selected, outputs one of the home interfaces described herein (e.g., home GUIs depicted in FIGS. 5C, and 5F to 5I-2); (2) a selectable insights icon which, upon being selected, outputs interfaces comprising reports, logbooks, daily summary data, including but not limited to daily graphs (e.g., outputs insights GUI 800, as shown in FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., profile GUI 600, as shown in FIG. 6A).

Referring to FIG. 6F, in some embodiments, help GUI 635 comprises one or more of the following selectable sections related to tutorials: (1) a sensor application tutorial section 636 (e.g., “How to Apply a Sensor”); (2) a sensor scanning tutorial section 637 (e.g., “How to Scan a Sensor”) which, upon being selected, outputs a series of interfaces can be presented to assist the user in scanning/starting a new sensor; (3) a glucose readings tutorial section 638 (e.g., “Glucose Readings”) which, upon being selected, outputs a series of tutorial interfaces relating to glucose readings.; and (4) an alarms tutorial section 639 (e.g., “Alarms”) which, upon being selected outputs a series of the tutorial interfaces relating to alarms.

In some embodiments, and still with reference to FIG. 6F, help GUI 635 further comprises one or more of the following selectable sections related to guides: (1) a quick start guide section 640; (2) a quick reference guide section 641; and (3) a user's manual section 642.

According to some embodiments, upon the user selecting the sensor application tutorial section 636, the quick start guide section 640, the quick reference guide section 641, or user's manual section 642 on help GUI 635 (FIG. 6F), a bottom sheet 648 (FIG. 6G) is displayed on help GUI 635, wherein the bottom sheets 648 comprises a plurality of selectable sensor type options 649. More specifically, and as depicted in FIG. 6G, in some embodiments, the plurality of selectable sensor type options 649 includes a selectable sensor type option corresponding to a first sensor type 649a (e.g., a textual description of a brand name associated with the first sensor type) and a selectable sensor type option corresponding to a second sensor type 649b (e.g., a textual description of a brand name associated with the second sensor type). Even more specifically, in some embodiments, upon the user selecting the sensor type option corresponding to the first sensor type 649a, a series of tutorial interfaces corresponding to the first sensor type can be presented to assist the user in applying the first sensor type to the user if bottom sheet 648 was outputted in response to the user selecting the sensor application tutorial section 636. Further, in some embodiments, upon the user selecting the sensor type option corresponding to the first sensor type 649a, a GUI comprising a quick start guide relating to the first sensor type can be outputted if bottom sheet 648 was outputted in response to the user selecting the quick start guide section 640. According to some embodiments, upon the user selecting the sensor type option corresponding to the first sensor type 649a, a GUI comprising a quick reference guide relating to the first sensor type can be outputted if bottom sheet 648 was outputted in response to the user selecting the quick reference guide section 641. According to some embodiments, upon the user selecting the sensor type option corresponding to the first sensor type 649a, a GUI comprising a user's manual relating to the first sensor type can be outputted if bottom sheet 648 was outputted in response to the user selecting the user's manual section 642.

In like manner, in some embodiments, upon the user selecting the sensor type option corresponding to the second sensor type 649b, a series of tutorial interfaces corresponding to the second sensor type can be presented to assist the user in applying the second sensor type to the user if bottom sheet 648 was outputted in response to the user selecting the sensor application tutorial section 636. Further, in some embodiments, upon the user selecting the sensor type option corresponding to the second sensor type 649b, a GUI comprising a quick start guide relating to the second sensor type can be outputted if bottom sheet 648 was outputted in response to the user selecting the quick start guide section 640. According to some embodiments, upon the user selecting the sensor type option corresponding to the second sensor type 649b, a GUI comprising a quick reference guide relating to the second sensor type can be outputted if bottom sheet 648 was outputted in response to the user selecting the quick reference guide section 641. According to some embodiments, upon the user selecting the sensor type option corresponding to the second sensor type 649b, a GUI comprising a user's manual relating to the second sensor type can be outputted if bottom sheet 648 was outputted in response to the user selecting the user's manual section 642.

According to some embodiments, the series of tutorial interfaces, the quick start guide, the quick reference guide, and user's manual corresponding to the first sensor type are different from the series of tutorial interfaces, the quick start guide, the quick reference guide, and user's manual corresponding to the second sensor type.

Referring to FIG. 6F, in some embodiments, help GUI 635 further comprises one or more of the following selectable sections related to safety: (1) a safety information section 643 which, upon being selected, outputs an interface comprising safety information related to the selected sensor type (e.g., a first sensor type or second sensor type), wherein the safety information related to the first sensor type can be different from the safety information related to the second sensor type; (2) an operating system compatibility section 644 which, upon being selected, outputs a compatibility GUI indicating that the operating system has been tested and is compatible with the App, wherein the compatibility GUI comprises a link to display a compatibility guide; (3) a phone warnings section 645; and (4) a sensor compatibility section 646. Further, in some embodiment, help GUI 635 can comprise a selectable option related to support, such as, a customer service section 647 which, upon being selected, links to customer service information.

According to some embodiments, upon the user selecting the phone warnings section 645, a phone warnings GUI 655 (FIG. 6H) can be outputted, wherein phone warnings GUI 655 includes a notice 656 with information relating to how the user's operating system features may impact the ability to receive alarms. In some embodiments, the content displayed on the notice 656 varies depending on the user's operating system (e.g., whether the user is using an iOS or Android device). In some embodiments, phone warning GUIs can also be presented as part of a series of tutorial interfaces during an onboarding process of the analyte monitoring software application, wherein the phone warning GUIs are presented as a last step of the onboarding process in order to optimize the user interface workflow.

According to some embodiments, upon the user selecting the sensor compatibility section 646, a GUI 660 (FIG. 6I) can be outputted, wherein GUI 660 includes a country indicator 661 configured to indicate the user's country (e.g., “United States”), along with a disclaimer 662 indicating to the user that the App is only compatible with certain sensors and that the user may need to download a different application if the sensor is not compatible. In some embodiments, GUI 660 further includes reminder relating to the installation of the appropriate application (e.g., “Make sure you have installed the right app for your Sensor”). Further, in some embodiments, GUI comprises an icon, link, or button to display a GUI 665 (FIG. 6J), wherein GUI 665 comprises a sensor compatibility guide which includes information on the sensors compatible with the analyte monitoring software application. In some embodiments, GUI 665 comprises a first link 667 which outputs the sensor compatibility guide in a first language (e.g., an English version of the sensor compatibility guide) and a second link 668 which outputs the sensor compatibility guide in a second language (e.g., a Spanish version of the sensor compatibility guide).

Further, in some embodiments, though not illustrated, upon the user selecting the sensor compatibility section 646, a modal can be outputted which indicates that the sensor cannot be utilized with the current version of the analyte monitoring software application if the sensor is incompatible.

Example Embodiments of Alarm GUIs and Features Related thereto

Various example embodiments relating to alarming, alarm interfaces, alarm settings interfaces for analyte monitoring systems, with respect to a Silent Mode feature, and other related features will now be described. In some instances, a user of an analyte monitoring software application may be in an environment in which the user desires to temporarily silence alarms (e.g., a movie theater, business meeting, quiet area, etc.). Several example embodiments described herein include a Silent Mode feature of an analyte monitoring software application that can be enabled for a predetermined period of time in order to temporarily prevent alarms from outputting an audible noise. In some embodiments, the alarms can continue to output vibratory alerts. In other embodiments, enabling Silent Mode can prevent any type of alarm output.

FIGS. 7A through 7M depict block diagrams of example embodiments of alarm interfaces, or features related thereto, any of which can be utilized with the embodiments described herein. It will be understood by those of skill in the art that any one or more of the example embodiments of the methods, interfaces, and systems described herein can either be implemented independently, or in combination with any of the other embodiments described in the present application.

Referring to FIG. 7A, an example embodiment of an alarm setting GUI 700 for an analyte monitoring software application is depicted, wherein the alarm setting GUI 700 comprising four selectable alarm options: an urgent low glucose alarm option 701, a low glucose alarm option 702, a high glucose alarm option 703, and a signal loss alarm option 704. Adjacent to each selectable alarm option is a textual indicator to indicate whether the alarm is on or off. Those of skill in the art will understand that, instead of a toggle, GUI 510 can include any one or more of: an on-off checkbox, on-off slider switch, on-off radio buttons, on-off buttons, and the like. Additionally, in some embodiments, a selectable “Learn More” option for additional information is provided beneath the selectable alarms. Details of the selectable alarm options, settings, and features related thereto, such as a Silent Mode feature, can be found in U.S. Publ. No. 2022/0248988, 2021/0378601, 2019/0183393, and Appendix A, which are hereby incorporated in their entirety for all purposes. According to some embodiments, alarm settings GUI 700 can be outputted in response to the user selecting the alarm icon on the bottom bar navigation menu displayed on any of the interfaces described herein.

In some embodiments, alarm settings GUI 700 further comprises a bottom bar navigation menu comprising a selectable home icon which, upon being selected, outputs one of the home interfaces described herein (e.g., home GUIs depicted in FIGS. 5C, and 5F to 5I-2); (2) a selectable insights icon which, upon being selected, outputs interfaces comprising reports, logbooks, daily summary data, including but not limited to daily graphs (e.g., outputs insights GUI 800, as shown in FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., profile GUI 600, as shown in FIG. 6A).

Further, as depicted in FIG. 7A, alarm settings GUI 700 includes a Silent Mode feature 705. In many embodiments, the Silent Mode feature 705 includes a switch 706 that can be toggled between an “on” state and an “off” state. Upon toggling switch 706 to the “on” state, a configuration modal 707, as seen in FIG. 7B, is displayed to the user. The user can specify the amount of time for Silent Mode to be enabled. In some embodiments, configuration modal 707 can have a maximum time limit such that the user cannot enable Silent Mode for longer than such (e.g., a maximum time limit of six hours). In some embodiments, configuration modal 707 can have a minimum time limit such that the user cannot enable Silent Mode for shorter than such (e.g., a minimum time limit of five minutes). In some embodiments, configuration modal 707 can include a “Save” button 708, which will save the user's selection in terms of the configurable duration of Silent Mode. Upon actuating “Save” button 708, the user can be prompted with another confirmation modal 712, as shown in FIG. 7C, which indicates to the user that “all glucose and signal loss alarms will be silenced” for the selected amount of time. In many embodiments, the confirmation modal 712 can include a “Turn On” option 713 to proceed with enabling Silent Mode, and/or a “Cancel” option, in case the user does not want to proceed with Silent Mode.

Although not depicted, in some embodiments, alarm settings GUI 700 can also prompt the user for authentication before enabling Silent Mode. In some embodiments, for example, the authentication step can comprise prompting the user to enter a password or PIN number. In some embodiments, the authentication step can comprise a biometric routine, such as a facial recognition routine performed by the operating system of the reader device. In some embodiments, the authentication step can be performed in response to the toggling of switch 706 from an “off” state to an “on” state. In some embodiments, the authentication step can be performed in response to the user actuating the “Save” button 708 or “Turn On” button 713, as shown in FIGS. 7B and 7C, respectively.

Referring next to FIGS. 7D to 7F, alarm settings GUI 700 is depicted, wherein alarm settings GUI 700 includes an in-app banner notification 709 to indicate to the user that Silent Mode is enabled until an expiration time (e.g., “Alarms Silenced until 3:41 pm”). In other embodiments, the in-app banner notification 709 can indicate to the user how much time is remaining until Silent Mode expires (e.g., “Alarms Silenced for 2 hours and 30 minutes”).

Referring to FIG. 7E, according to another aspect of some embodiments, if the user attempts to configure the alarms (e.g., by selecting “Low Glucose Alarm”), modal 710 can be displayed to the user to indicate that alarms cannot be customized during Silent Mode, as shown in FIG. 7E. In some embodiments, modal 710 can include a selectable “Turn Off” button, to allow the user to immediately disable Silent Mode.

In some embodiments, in-app banner notification 709 can also be selectable and configured to display, upon selection by the user, a modal 711 to provide further information to the user regarding Silent Mode, as shown in FIG. 7F. In some embodiments, modal 711 can also include a “Turn Off” button, to allow the user to immediately disable Silent Mode.

Referring next to FIG. 7G, an example embodiment of a lock screen GUI 740 is depicted, wherein a Silent Mode notification 741 is displayed to the user to indicate that Silent Mode is enabled. In some embodiments, Silent Mode notification 741 indicates to the user that Silent Mode is enabled until an expiration time (e.g., “Alarms Silenced until 10:41 am” or “Silent Mode is on until 10:41 am”). In other embodiments, Silent Mode notification 741 can indicate to the user how much time is remaining until Silent Mode expires (e.g., “Alarms Silenced for 2 hours and 30 minutes”). In some embodiments, Silent Mode notification 741 indicates to the user that alarms will be delivered without sound because silent mode is on (e.g., “Just a reminder: Silent Mode is on, and Alarms will be delivered without sound.”).

Still referring to FIG. 7G, in some embodiments, the Silent Mode notification 741 can be periodically outputted to the lock screen GUI 740 throughout the duration of the Silent Mode period (e.g., every 30 minutes, every hour, every two hours, etc.). In other embodiments, the Silent Mode notification 741 can be outputted to the lock screen GUI 740 in response to one or more predetermined events (e.g., user closing or placing the analyte monitoring software application in the background). In some embodiments, the Silent Mode notification 741 can be outputted to the lock screen GUI 740 after a predetermined period of time has elapsed before the expiration time. For example, in some embodiments, the Silent Mode notification is outputted after half the duration of expiration time has elapsed, or when half the expiration time of Silent Mode is remaining (e.g., if the alarms are silenced for one hour, then the Silent Mode notification 741 is outputted 30 minutes after Silence Mode is turned on). Those of skill in the art will recognize that the Silent mode notification 741 can be outputted after various other durations of time have elapsed prior to the expiration time (e.g., when 25% of the time has elapsed prior to expiration time, when 75% of the time has elapsed prior to expiration time), without departing from the scope of the present disclosure.

According to an aspect of the embodiments, and with reference to FIG. 7G, the Silent Mode notification 741 can be outputted silently to the lock screen GUI 740. In some embodiments, the Silent Mode notification 741 is outputted silently to the lock screen GUI 740 even when all other device notifications external to the analyte monitoring software application present auditory outputs. In some embodiments, the Silent Mode notification 741 can present a vibratory output when outputted to the lock screen GUI 740.

According to another aspect of some embodiments, the analyte monitoring software application can be configured to provide escalating alarms. In particular, during an initial alarm presentation, the analyte monitoring software application can present a vibratory output. Then, for each subsequent alarm presentation, the analyte monitoring software application can present an auditory output at an increased volume. According to some embodiments, the subsequent alarm presentations can continue to output at an increased volume at a predetermined interval until reaching a maximum volume. In some embodiments, for example, the volume of the alarm can be increased every 10 seconds, every 30 seconds, every minute, etc.

Additionally, in some embodiments, an alarm settings GUI can include an interface such that the user can enable, disable, and configure escalating alarms. For example, in some embodiments, the user can select the interval at which the volume of the escalating alarm increases. In other embodiments, the user can select a specific sound (or sound file) to utilize with the escalating alarm mode. In still other embodiments, the escalating alarm setting can be an optional parameter associated with each of the alarms (e.g., Low Glucose Alarm, High Glucose Alarm, Signal Loss Alarm). In this regard, the user can utilize escalating alarms only for specific alarm conditions. In still other embodiments, the maximum volume of an escalating alarm can depend on whether the analyte monitoring software application has an Override Do-No-Disturb (“Override DND”) setting enabled. In some embodiments, for example, if the Override DND setting is enabled, then the maximum volume would be the volume to which the reader device's volume is currently set. On the other hand, in some embodiments, if the Override DND setting is disabled, the maximum volume can be a preset or default volume set by the analyte monitoring software application. In some embodiments, the auditory output associated with the escalating alarm can be a preset tone or a custom tone.

According to some embodiments, the Override DND setting is turned on by default for optional glucose alarms (e.g., low glucose alarm and high glucose alarm). In this manner, the user will the optional glucose alarms regardless of the device's auditory, vibratory, or DND settings. If the user wishes to have the optional glucose alarms follow the device's auditory and vibratory setting, the user must turn off Override DND from the analyte monitoring software application for each alarm.

According to some embodiments, the Override DND setting is turned on by default for the optional signal loss alarm. In this manner, the user will the optional glucose alarms regardless of the device's auditory, vibratory, or DND settings. If the user wishes to have the optional glucose alarms follow the device's auditory and vibratory setting, the user must turn off Override DND from the analyte monitoring software application.

According to another aspect of the embodiments, mandatory system alarms (e.g., alarms related to sensor termination, such as, replace sensor alarms, sensor ended alarms, check sensor alarms for the second sensor type, and app stopped alarms) cannot be turned off or modified and will always sound regardless of the device's auditory and vibratory or DND settings.

FIGS. 7H and 7I are example embodiments of GUIs comprising alarms for use with an analyte monitoring software application. FIG. 7H, for example, is a GUI 70 depicting an alarm for an analyte monitoring software application, wherein the alarm comprises one or more of the following: an alarm condition text 756 (e.g., “Low Glucose Alarm”), an analyte level measurement 757 (e.g., a current glucose level of “67 mg/dL”) associated with the alarm condition, an alarm icon 753 adjacent to the alarm condition text 756, a trend indicator 758 (e.g., a trend arrow or directional arrow) associated with the alarm condition, and an alert condition tag 759 comprising textual information on an alert condition associated with the alarm condition (e.g., “Critical”). For example, in some embodiments the alert condition tag 758 can be presented to indicate that the alarm is a critical alert (FIG. 7H). Specifically, in some embodiments, the alarm can be presented as a critical alert when the user enables Override DND for an alert type and Critical Alerts features are enabled. In other embodiments, and as depicted in FIG. 7I, the alert condition tag 754 can be presented to indicate that the alarm is time sensitive. Specifically, in some embodiments, the alarm can be presented as time sensitive when the user has disabled Override DND for an alarm type and the Time Sensitive notification feature is enabled at the operating system level. More specifically, in some embodiments, if the Critical Alerts features are disabled, a signal loss alarm will be provided as a time sensitive alarm if time sensitive notifications are enabled at the operating system level.

Referring next to FIG. 7J, informational blocked app GUI 760 for an analyte monitoring software application is depicted. Information blocked app GUI 760 is similar to information blocked app GUI 425 (FIG. 4I), except that it further comprises an in-app banner notification 761, wherein in-app banner notification 761 indicates that alarms are unavailable.

Turning next to FIG. 7K, another example embodiment of an informational blocked app GUI 765 for an analyte monitoring software application is depicted. In some embodiments, for example, an informational blocked app GUI 765 can be presented in response to the determination that the Bluetooth functionality on the reader device has been disabled. Informational blocked app GUI 765 can indicate to the user that Bluetooth must be enabled in order to utilize the analyte monitoring software application. Further, in some embodiments, and as depicted in FIG. 7K, informational blocked app GUI 765 comprises an in-app banner notification 766 indicating that alarms are unavailable. According to some embodiments, informational blocked app GUI 765 further comprises a Bluetooth enabling button 767 which, upon being selected, outputs a confirmation modal 768 (FIG. 7L) which comprises a selectable deny button and a selectable settings button. Specifically, upon the user selecting the settings button on confirmation modal 768, the user is navigated to another GUI configured to allow the user to enable the BLE functionality of the reader device, such as a settings GUI that is part of the operating system of the reader device. In some embodiments, confirmation modal 768 comprises a selectable allow button instead of a settings button which, upon being selected, allows the user to enable BLE functionality.

Turning to FIG. 7M, alarm setting GUI 700 is depicted with one or more notifications notification associated with the detected one or more alarm unavailability conditions. Specifically, the one or more notifications can comprise a modal window 769, as seen in FIG. 7M, wherein the modal 769 can provide a number of possible reasons for the alarm unavailability condition along with a “Settings” button that opens up the corresponding settings interface to allow the user to correct the condition. In still other embodiments, the modal can present a specific alarm that is unavailable, the reason for the alarm unavailability condition, and a “Settings” button that opens up the corresponding settings interface to allow the user to correct the condition. These examples are meant to be illustrative only, and those of skill in the art will recognize that other combinations and permutations of modals can be implemented and are fully within the scope of the present disclosure.

According to another aspect of the embodiments, the one or more notifications associated with the detected one or more alarm unavailability conditions can comprise an in-app notification 769 within an analyte monitoring software application, as seen alarm setting GUI 700 depicted in FIG. 7M. In some embodiments, the notification associated with the alarm unavailability condition can be presented as an in-app banner notification 769. Although not shown, in some embodiments, the in-app banner notification 769 can persist through different interfaces within the analyte monitoring software application (e.g., home, reports, and/or logbook interfaces, etc.). Further, in some embodiments, the in-app banner notification 769 is fixed to a predetermined position on an interface (e.g., on a top portion of alarm settings GUI 700) such that if the interface is scrollable, the in-app banner notification is fixed in positioned so as to appear on the same portion of the interface regardless of whether the other portions of the interface are movable, or whether the interface is scrolled up or down.

### Example Embodiments of Insights GUIs and Features Related Thereto

FIGS. 8A through 8B are block diagrams depicting example embodiments of insights interfaces, or features related thereto, for an analyte monitoring software application of an analyte monitoring system, any of which can be utilized with the embodiments described herein. Referring to FIGS. 8A and 8B, an example embodiment of a block diagram of an insights GUI 800 for an analyte monitoring software application is depicted. According to some embodiments, insights GUI 800 can be outputted in response to the user selected the insights icon on the bottom bar navigation menu displayed on any of the interfaces described herein.

With reference to FIGS. 8A and 8B, insights GUI 800 configured to output a daily summary related to the data indicative of the analyte level, and provide daily updates and information related to analyte data for a particular period of time (e.g., a particular day). Specifically, insights GUI 800 comprises a Daily Summary view 801a or first view 801a, (FIG. 8A) and a Reports view 801b or second view 801b (FIG. 8B), with a toggle, switch, or slidable element that allows the user to select or transition between the two views.

According to one aspect of the embodiments, and with particular reference to FIG. 8A, the Daily Summary view 801a is configured to output information related to analyte data, trends, historical information, and/or alarms on a single screen. Further, the Daily Summary view 801a further comprises a date indicator 803 (e.g., “Thurs, March 10”), showing the relevant date associated with the displayed data/information. In some embodiments, a selectable left switch 805a is displayed adjacent to and to the left of the date indicator 803, and a selectable right switch 805b is displayed adjacent to and to the right of the date indicator 803. Specifically, the selectable left switch 805a, upon being selected, updates the relevant date associated with the daily updates and information outputted to insights GUI 800. More specifically the selectable left switch 805a changes the relevant date to a preceding day and the date indicator 803 is updated accordingly to reflect the relevant preceding day's date (e.g., upon the user selecting the left switch 805a, the date indicator 803 shows “Wed March 9” instead of “Thurs March 10”). Similarly, the selectable right switch 805b, upon being selected, updates the relevant date associated with the daily updates and information outputted to insights GUI 800. More specifically the selectable right switch 805b changes the relevant date to a subsequent day and the date indicator 803 is updated accordingly to reflect the relevant preceding day's date (e.g., upon the user selecting the right switch 805b, the date indicator 803 shows “Fri March 11” instead of “Thurs March 10”).

Further, and still with reference to FIG. 8A, the Daily Summary view 801a is configured to output an analyte graph summary section 807 (e.g., a glucose graph summary section 807) and a logbook section 808. In some embodiments, the logbook section 808 is displayed directly adjacent to and below the analyte graph summary section 807. Specifically, the analyte graph summary section 807 comprises an analyte graph 810 reflecting historical analyte data and current analyte data (if applicable) for a particular day associated with the insights GUI 800. More specifically, the analyte graph 810 can comprise an analyte trend line 811 to reflect the user's analyte level, based on the data indicative of the analyte level, over the particular day associated with the insights GUI 800. In some embodiments, the analyte graph 810 can include a shaded color area (e.g., a green colored portion) 812 to indicate the user's target analyte range (e.g., target glucose threshold) associated with the user's analyte level.

In some embodiments, and still with reference to FIG. 8A, the analyte graph summary section 907 can further comprise an event timeline 813. Specifically, in some embodiments, the event timeline 813 can be positioned below, above, or adjacent to the analyte graph 810. More specifically, one or more icons 814 can be positioned the event timeline 813, and can be oriented such that each icon 814 aligns with one or more points along an x-axis of the analyte graph 810. Each of the one or more icons 814 is configured to represent the occurrence of an event for the user. In this manner, the user can visually associate an event represented by each of the one or more icons 814 with the user's analyte level that is represented by a corresponding point on the analyte trendline 811. In some embodiments, the one or more icons 814 can include: a food icon, a rapid-acting insulin icon, a long-acting insulin icon, and an exercise icon. In some embodiments, if one or more events occurred at a same time, then one icon 814 is used to represent the one or more events. Specifically, in some embodiments, the one icon 814 used to represent the one or more events can include a numerical number to represent the number of events represented by the one icon 814 (e.g., “2” to represent the occurrence of two contemporaneous events).

With reference to FIG. 8A, the logbook section 808 can comprise one or more activity events related to user's analyte data. Specifically, the activity events can include one or more of the following: (1) one or more meal events 815 configured to indicate a time of a logged or detected meal, (2) one or more treatment events 816 configured to indicate a time in which the user administered a treatment, and/or (3) one or more exercise events 818 configured to indicate a time of a logged or detected exercise. Specifically, in some embodiments, each of the one or more meal events 815 can include one or more of the following: (1) a timestamp 817 associated with a time of the meal event 815 (e.g., 8:45 PM PST″); and (2) a macronutrient counter 821 (e.g., a carbohydrate counter 821), wherein the macronutrient counter indicates the number of macronutrient associated with the meal event 815 (e.g., “25 g” to represent 25 grams of carbohydrates associated with the meal event 815). Those of skill in the art will appreciate that various other types of information can be displayed with respect to each of the meal events 815, and that various other macronutrients can be represented by the macronutrient counter 821 without departing from the scope of the present disclosure.

Further, in some embodiments, and still with reference to FIG. 8A, each of the one or more treatment events 816 can be a rapid-acting insulin event 816a or a long-lasting insulin event 816b. According to an aspect of the embodiments, each of the one or more treatment events 816 can comprise a textual description 822 which indicates the type of treatment event 816 (e.g., “rapid-acting insulin” or “long-lasting insulin”), a timestamp 820 associated with a time of the treatment event 816 (e.g., 9:03 PM PST″), and a dosage counter 823. Specifically, the dosage counter 823 is configured to indicate a dosage associated with the treatment event 816. For example, if the user had 10 units of rapid-acting insulin, then the corresponding dosage counter 823 can indicate “10 units” were utilized (e.g., “10U”). Those of skill in the art will appreciate that various other types of treatment events and information related thereto can be displayed on insights GUI 800 without departing from the scope of the present disclosure.

According to another aspect of the embodiments, each of the one or more exercise events 818 is configured to represent a time in which an exercise was detected logged by the user. Each exercise event 818 can comprise a timestamp 824 associated with a time of the exercise event 818 along with an exercise duration indicator 826 configured to indicate a duration or amount of time spend on the exercise represented by the exercise event 818.

In some embodiments, the one or more meal events 815, one or more treatment events 816, and/or one or more exercise events 818, can be displayed in chronological order in the logbook section 808.

Turning to FIG. 8B, the reports view 801b of insights GUI 800 is depicted. Specifically, reports view 801b is configured to output a plurality of selectable report options, including but not limited to: (1) a Time-In-Ranges report option 830; (2) an average analyte report option 831 (e.g., an average glucose report option); (3) an events report option 832 (e.g., a low glucose events report option); (4) a daily patterns report option 833; and (5) a glucose management indicator (GMI) report option 834. Though not illustrated, each of the selectable report options is configured to output interfaces comprising information relating to their respective report.

According to another aspect of the embodiments, and as best depicted in FIG. 8B, the insights GUI 800 can further comprise a bottom bar navigation menu comprising: a selectable home icon which, upon being selected, outputs one of the home interfaces described herein (e.g., home GUIs depicted in FIGS. 5C, and 5F to 5I-2); (2) a selectable insights icon which, upon being selected, outputs insights GUI 800 (FIG. 8A or 8B); (3) a selectable alarms icon which, upon being selected, outputs interfaces relating to alarms (e.g., alarm settings GUI 700, as shown in FIG. 7A); and (4) a selectable profile icon which, upon being selected, outputs an interface related to the user's profile or account (e.g., profile GUI 600, as shown in FIG. 6A).

Details regarding insights GUIs, reports, time-in-range, logbook, daily summary, and event features are described in U.S. Publ. Nos. 2019/0183393 and 2021/0282672, U.S. patent application Ser. No. 18/380,590, filed on Oct. 16, 2023, and U.S. patent application Ser. No. 18/394,684, filed on Dec. 22, 2023, which are hereby incorporated by reference in their entirety for all purposes.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Exemplary embodiments of the invention are set forth in the following numbered clauses:
1. A glucose monitoring system, comprising:
   a sensor control device comprising a glucose sensor coupled with sensor electronics, the sensor control device configured to transmit data indicative of a glucose level; and
   a reader device comprising a display, wireless communication circuitry coupled with one or more processors, one or more processors coupled with a first memory, the first memory storing a glucose monitoring software application that, when executed by the one or more processors, cause the one or more processors to:
      cause transmission of a scan response from the sensor control device to the reader device according to a first wireless communication protocol; and
      determine, based on the scan response, whether the glucose sensor is a first sensor type or a second sensor type, wherein the glucose monitoring software application is configured to be modified to a first configuration if the glucose sensor is determined to be the first sensor type, and wherein the glucose monitoring software application is further configured to be modified to a second configuration if the glucose sensor is determined to be the second sensor type.
2. The glucose monitoring system of clause 1, wherein in the first configuration, the glucose monitoring software application, when executed by the one or more processors, further causes the one or more processors to:
   establish, between the sensor control device and the reader device, a wireless communication link according to a second wireless communication protocol, wherein the reader device is configured to receive the data indicative of the glucose level according to the second wireless communication protocol.
3. The glucose monitoring system of clause 1 or 2, wherein the reader device is further configured to receive the data indicative of the glucose level according to the first wireless communication protocol.
4. The glucose monitoring system of clause 2, wherein the second wireless communication protocol is different than the first wireless communication protocol.
5. The glucose monitoring system of clause 2 or 4, wherein the second wireless communication protocol is a Bluetooth or Bluetooth Low Energy protocol, and wherein the first wireless communication protocol is an NFC protocol.
6. The glucose monitoring system of any preceding clause, wherein the reader device is configured to receive the data indicative of the glucose level at a predetermined interval.
7. The glucose monitoring system of clause 6, wherein the predetermined interval is every minute.
8. The glucose monitoring system of any preceding clause, wherein in the second configuration, the glucose monitoring software application, when executed by the one or more processors, further causes the one or more processors to:
   establish, between the sensor control device and the reader device, a wireless communication link according to a second wireless communication protocol, wherein the reader device is configured to receive the data indicative of the glucose level according to the second wireless communication protocol.
9. The glucose monitoring system of clause 8, wherein the second wireless communication protocol is different than the first wireless communication protocol.
10. The glucose monitoring system of clause 8 or 9, wherein the second wireless communication protocol is a Bluetooth or Bluetooth Low Energy protocol, and wherein the first wireless communication protocol is an NFC protocol.
11. The glucose monitoring system of clause 8, 9 or 10, wherein the reader device is configured to receive the data indicative of the glucose level at a predetermined interval.
12. The glucose monitoring system of clause 11, wherein the predetermined interval is every minute.
13. The glucose monitoring system of any preceding clause, wherein in the first configuration, the glucose monitoring software application, when executed by the one or more processors, further causes the one or more processors to:
   output a graphical user interface (GUI) comprising a first sensor type indicator, wherein the first sensor type indicator comprises a textual description of a brand name associated with the first sensor type.
14. The glucose monitoring system of any preceding clause, wherein in the second configuration, the glucose monitoring software application, when executed by the one or more processors, further causes the one or more processors to:
   output a graphical user interface comprising a second sensor type indicator, wherein the second sensor type indicator comprises a textual description of a brand name associated with the second sensor type.
15. The glucose monitoring system of any preceding clause, wherein in the first configuration, the glucose monitoring software application, when executed by the one or more processors, further causes the one or more processors to:
   cause autonomous communication of the data indicative of the glucose level at a predetermined interval between the sensor control device and the reader device or cause communication of the data indicative of the glucose level according to the first wireless communication protocol; and
   in response to a reconnection through the first wireless communication protocol following an interruption of a communication link between the sensor control device and the reader device, the reader device is configured to request historical data indicative of the glucose level from the sensor control device, wherein the historical data indicative of the glucose level is associated with a specific lifecount range; and
   upon receiving the request, the sensor control device is further configured to retrieve the requested historical data indicative of the glucose level from a second memory and transmit the requested historical data indicative of the glucose level to the reader device, wherein the reader device is further configured to store the transmitted historical requested historical data indicative of the glucose level in the first memory.
16. The glucose monitoring system of clause 15, wherein the lifecount range includes up to a last eight hours of historical data indicative of the glucose level.
17. The glucose monitoring system of clause 15 or 16, wherein the first wireless communication protocol is an NFC protocol.
18. The glucose monitoring system of any preceding clause, wherein in the second configuration, the glucose monitoring software application, when executed by the one or more processors, further causes the one or more processors to:
   cause autonomous communication of the data indicative of the glucose level at a predetermined interval between the sensor control device and the reader device; and
   in response to a reconnection following an interruption of a communication link between the sensor control device and the reader device, the reader device is configured to request historical data indicative of the glucose level from the sensor control device according to a lifecount metric; and
   upon receiving the request, the sensor control device is further configured to retrieve the requested historical data indicative of the glucose level from a second memory and transmit the requested historical data indicative of the glucose level to the reader device, wherein the reader device is further configured to store the transmitted historical requested historical data indicative of the glucose level in the first memory.
19. The glucose monitoring system of clause 18, wherein the lifecount metric comprises a numerical value configured to be incremented and tracked on the reader device, wherein the lifecount metric is configured to be indicative of an amount of time elapsed since activation of the sensor control device.
20. The glucose monitoring system of clause 18 or 19, wherein the data indicative of the glucose level is autonomously communicated between the sensor control device and the reader device according to a second wireless communication protocol.
21. The glucose monitoring system of clause 20, wherein the second wireless communication protocol is a Bluetooth or Bluetooth Low Energy protocol, and wherein the first wireless communication protocol is an NFC protocol.
22. The glucose monitoring system of any preceding clause, wherein the first configuration is different from the second configuration.
23. The glucose monitoring system of any preceding clause, wherein the first sensor type is different from the second sensor type.
24. The glucose monitoring system of any preceding clause, wherein in the first configuration, the glucose monitoring software application, when executed by the one or more processors, further causes the one or more processors to:
   output a first set of GUIs corresponding to the first sensor type.
25. The glucose monitoring system of any preceding clause, wherein in the second configuration, the glucose monitoring software application, when executed by the one or more processors, further causes the one or more processors to:
   output a second set of GUIs corresponding to the second sensor type.
26. A glucose monitoring system, comprising:
   a sensor control device comprising a glucose sensor coupled with sensor electronics, the sensor control device configured to transmit data indicative of a glucose level; and
   a reader device comprising a display, wireless communication circuitry coupled with one or more processors, one or more processors coupled with a memory, the memory storing a glucose monitoring software application that, when executed by the one or more processors, cause the one or more processors to:
      cause transmission of a scan response from the sensor control device to the reader device according to a first wireless communication protocol;
      determine, based on the scan response, whether the glucose sensor is a first sensor type or a second sensor type;
      establish between the sensor control device and the reader device, a wireless communication link according to a second wireless communication protocol, wherein the reader device is configured to receive the data indicative of the glucose level according to the second wireless communication protocol; and
      output, based on the received data indicative of the glucose level, a home graphical user interface (GUI) comprising the received data indicative of the glucose level and corresponding to the first sensor type if it is determined that the glucose sensor is the first sensor type or the received data indicative of the glucose level and corresponding to the second sensor type if it is determined that the glucose sensor is the second sensor type.
27. The glucose monitoring system of clause 26, wherein the home GUI further
   comprises a first portion with a numeric representation of a current glucose concentration value, a directional trend arrow configured to indicate a glucose trend direction, and a text description configured to provide contextual information related to the data indicative of the glucose level.
28. The glucose monitoring system of clause 27, wherein the home GUI further
   comprises a second portion with a glucose trend graph comprising a glucose trend line, wherein the glucose trend line is configured to reflect historical data indicative of the glucose level over time, the glucose trend line comprising a current glucose data point configured to indicate a current glucose concentration value.
29. The glucose monitoring system of clause 28, wherein the glucose trend graph is configured to be interactive.
30. The glucose monitoring system of clause 28 or 29, wherein the home GUI further comprises a third portion comprising a graphical indicator and textual information configured to represent a remaining amount of sensor life.
31. The glucose monitoring system of clause 30, wherein the graphical indicator comprises a numerical value corresponding to a number of data, hours, or minutes remaining in a lifetime of the sensor, and a tab comprising a colored portion indicative of a status of the remaining lifetime of the sensor
32. The glucose monitoring system of any of clauses 26 to 30, wherein the home GUI further comprise a sensor type indicator configured to indicate whether the glucose sensor is the first sensor type or the second sensor type.
33. The glucose monitoring system of clause 32, wherein the sensor type indicator comprises a textual description of a brand name associated with the first sensor type if it is determined the glucose sensor is the first sensor type, and wherein the sensor type indicator comprises a textual description of a brand name associated with the second sensor type if it is determined the glucose sensor is the second sensor type.
34. The glucose monitoring system of any of clauses 26 to 33, wherein the home GUI further comprises a bottom bar navigation menu with a plurality of selectable icons.
35. The glucose monitoring system of clause 34, wherein the plurality of selectable icons comprises a selectable home icon which, upon being selected outputs the home GUI.
36. The glucose monitoring system of clause 34 or 35, wherein the plurality of selectable icons comprises a selectable insights icon which, upon being selected outputs an insights GUI configured to reflect insights, reports, logbooks, or daily summary data related to the data indicative of the glucose level.
37. The glucose monitoring system of clause 34, 35 or 36, wherein the plurality of selectable icons comprises a selectable profile icon which, upon being selected outputs a GUI related to the user's profile or account on the glucose monitoring software application.
38. The glucose monitoring system of any of clauses 34 to 37, wherein the plurality of selectable icons comprises a selectable alarms icon which, upon being selected outputs a GUI related to alarms.
39. The glucose monitoring system of any of clauses 26 to 38, wherein the first sensor type is different than the second sensor type.
40. The glucose monitoring system of any of clauses 26 to 39, wherein the first wireless communication protocol is different from the second wireless communication protocol if it is determined that the glucose sensor is the second sensor type.
41. The glucose monitoring system of clause 40, wherein the first wireless communication protocol is an NFC protocol, and wherein the second wireless communication protocol is a Bluetooth or Bluetooth Low Energy protocol.
42. The glucose monitoring system of any of clauses 26 to 41, wherein the first wireless communication protocol is different from the second wireless communication protocol if it is determined that the glucose sensor is the first sensor type.
43. The glucose monitoring system of clause 42, wherein the first wireless communication protocol is an NFC protocol, and wherein the second wireless communication protocol is a Bluetooth or Bluetooth Low Energy protocol.
44. The glucose monitoring system of any of clauses 26 to 43, wherein the reader device is further configured to receive the data indicative of the glucose level according to the first wireless communication protocol if it is determined that the glucose sensor is the first sensor type.
45. The glucose monitoring system of clause 44, wherein the first wireless communication protocol is an NFC protocol, and wherein the second wireless communication protocol is a Bluetooth or Bluetooth Low Energy protocol.
46. A glucose monitoring system, comprising:
   a sensor control device comprising a glucose sensor coupled with sensor electronics, the sensor control device configured to transmit data indicative of a glucose level; and
   a reader device comprising a display, wireless communication circuitry coupled with one or more processors, one or more processors coupled with a memory, the memory storing a glucose monitoring software application that, when executed by the one or more processors, cause the one or more processors to:
      cause transmission of a scan response from the sensor control device to the reader device according to a first wireless communication protocol;
      determine, based on the scan response, whether the glucose sensor is a first sensor type or a second sensor type;
      establish between the sensor control device and the reader device, a wireless communication link according to a second wireless communication protocol, wherein the reader device is configured to receive the data indicative of the glucose level according to the second wireless communication protocol; and
      output, based on the received data indicative of the glucose level, a graphical user interface (GUI) configured to transition between a first view and a second view, wherein in the first view, the GUI is configured to output a daily summary related to the data indicative of the glucose level, and wherein in the second view the GUI is configured to output a plurality of selectable report options related to the data indicative of the glucose level.
47. The glucose monitoring system of clause 46, wherein the GUI comprises a toggle which, upon being selected, is configured to transition the GUI between the first view and the second view.
48. The glucose monitoring system of clause 46 or 47, wherein the first view comprises a data indicator configured to indicate a relevant date associated with the daily summary.
49. The glucose monitoring system of clause 46, 47 or 48, wherein the first view further comprises one or more switches configured to update the relevant date associated with the daily summary.
50. The glucose monitoring system of any of clauses 46 to 49, wherein the first view comprises a glucose graph summary section and a logbook section.
51. The glucose monitoring system of clause 50, wherein the glucose graph summary section comprises a glucose graph reflecting historical data indicative of the glucose level and current data indicative of the glucose level for a particular day associated with the daily summary.
52. The glucose monitoring system of clause 50 or 51, wherein the glucose graph summary section further comprises an event timeline with one or more icons positioned thereon, wherein each of the one or more icons is indicative of an occurrence of an event.
53. The glucose monitoring system of clause 52, wherein the one or more icons can include a food icon, a rapid-acting insulin icon, a long-lasting insulin icon, and an exercise icon.
54. The glucose monitoring system of clause 52 or 53, wherein each of the one or more icons is configured to align with one or more points along an x-axis of the glucose graph to indicate a corresponding point on the glucose graph associated with the each of the one or more icons.
55. The glucose monitoring system of any of clauses 50 to 54, wherein the logbook section comprises one or more activity events related to the data indicative of the glucose level.
56. The glucose monitoring system of clause 55, wherein the one or more activity events includes one or more meal events configured to indicate a time of a logged or detected meal, wherein each of the one or more meal events comprise a timestamp associated with the meal, and a macronutrient counter configured to indicate a number of macronutrients associated with the meal.
57. The glucose monitoring system of clause 56, wherein the macronutrient counter is a carbohydrate counter configured to indicate a number of carbohydrate grams associated with the meal.
58. The glucose monitoring system of clause 55, 56 or 57, wherein the one or more activity events includes one or more treatment events configured to indicate a time in which the user administered a treatment, wherein each of the one or more treatment events comprises a textual description configured to indicate a type of the treatment, a timestamp associated with a time of the treatment, and a dosage counter configured to indicate a dosage associated with the treatment.
59. The glucose monitoring system of clause 58, wherein the one or more treatment events is a rapid-acting insulin event or a long-lasting insulin event.
60. The glucose monitoring system of any of clauses 55 to 59, wherein the one or more activity events includes one or more exercise events configured to indicate a time of a logged or detected exercise, wherein each of the one or more exercise events comprise a timestamp associated with a time of the exercise and an exercise duration indicator configured to indicate a duration or amount of time spent on the exercise.
61. The glucose monitoring system of any of clauses 55 to 60, wherein the one or more activity events are displayed in the logbook section in chronological order.
62. The glucose monitoring system of any of clauses 46 to 61, wherein the GUI further comprises a bottom bar navigation menu with a plurality of selectable icons.
63. The glucose monitoring system of clause 62, wherein the plurality of selectable icons comprises a selectable home icon which, upon being selected outputs a home GUI reflecting the data indicative of the glucose level.
64. The glucose monitoring system of clause 62 or 63, wherein the plurality of selectable icons comprises a selectable insights icon which, upon being selected outputs the GUI in the first view or the second view.
65. The glucose monitoring system of clause 62, 63 or 64, wherein the plurality of selectable icons comprises a selectable profile icon which, upon being selected outputs a GUI related to the user's profile or account on the glucose monitoring software application.
66. The glucose monitoring system of any of clauses 62 to 65, wherein the plurality of selectable icons comprises a selectable alarms icon which, upon being selected outputs a GUI related to alarms.
67. The glucose monitoring system of any of clauses 46 to 67, wherein the first sensor type is different than the second sensor type.
68. The glucose monitoring system of any of clauses 46 to 67, wherein the first wireless communication protocol is different from the second wireless communication protocol if it is determined that the glucose sensor is the second sensor type.
69. The glucose monitoring system of clause 68, wherein the first wireless communication protocol is an NFC protocol, and wherein the second wireless communication protocol is a Bluetooth or Bluetooth Low Energy protocol.
70. The glucose monitoring system of any of clauses 46 to 69, wherein the first wireless communication protocol is different from the second wireless communication protocol if it is determined that the glucose sensor is the first sensor type.
71. The glucose monitoring system of clause 70, wherein the first wireless communication protocol is an NFC protocol, and wherein the second wireless communication protocol is a Bluetooth or Bluetooth Low Energy protocol.
72. The glucose monitoring system of any of clauses 46 to 71, wherein the reader device is further configured to receive the data indicative of the glucose level according to the first wireless communication protocol if it is determined that the glucose sensor is the first sensor type.
73. The glucose monitoring system of clause 72, wherein the first wireless communication protocol is an NFC protocol, and wherein the second wireless communication protocol is a Bluetooth or Bluetooth Low Energy protocol.
74. The glucose monitoring system of any of clauses 46 to 73, wherein the plurality of selectable report options comprises a time-in-range report option, an average glucose report option, an event report option, and a daily patterns report option, and a glucose management indicator report option.
75. The glucose monitoring system of clause 74, wherein the events report option is a low glucose events report option.

1. A glucose monitoring system, comprising:
a sensor control device comprising a glucose sensor coupled with sensor electronics, the sensor control device configured to transmit data indicative of a glucose level; and
a reader device comprising a display, wireless communication circuitry coupled with one or more processors, one or more processors coupled with a first memory, the first memory storing a glucose monitoring software application that, when executed by the one or more processors, cause the one or more processors to:
cause transmission of a scan response from the sensor control device to the reader device according to a first wireless communication protocol; and
determine, based on the scan response, whether the glucose sensor is a first senor type or a second sensor type, wherein the glucose monitoring software application is configured to be modified to a first configuration if the glucose sensor is determined to be the first sensor type, and wherein the glucose monitoring software application is further configured to be modified to a second configuration if the glucose sensor is determined to be the second sensor type.

2. The glucose monitoring system of claim 1, wherein in the first configuration, the glucose monitoring software application, when executed by the one or more processors, further causes the one or more processors to:
establish, between the sensor control device and the reader device, a wireless communication link according to a second wireless communication protocol, wherein the reader device is configured to receive the data indicative of the glucose level according to the second wireless communication protocol.

3. The glucose monitoring system of claim 2, wherein the reader device is further configured to receive the data indicative of the glucose level according to the first wireless communication protocol.

4. The glucose monitoring system of claim 3, wherein the second wireless communication protocol is different than the first wireless communication protocol.

5. The glucose monitoring system of claim 3, wherein the second wireless communication protocol is a Bluetooth or Bluetooth Low Energy protocol, and wherein the first wireless communication protocol is an NFC protocol.

6. The glucose monitoring system of claim 2, wherein the reader device is configured to receive the data indicative of the glucose level at a predetermined interval.

7. The glucose monitoring system of claim 6, wherein the predetermined interval is every minute.

8. The glucose monitoring system of claim 1, wherein in the second configuration, the glucose monitoring software application, when executed by the one or more processors, further causes the one or more processors to:
establish, between the sensor control device and the reader device, a wireless communication link according to a second wireless communication protocol, wherein the reader device is configured to receive the data indicative of the glucose level according to the second wireless communication protocol.

9. The glucose monitoring system of claim 8, wherein the second wireless communication protocol is different than the first wireless communication protocol.

10. The glucose monitoring system of claim 8, wherein the second wireless communication protocol is a Bluetooth or Bluetooth Low Energy protocol, and wherein the first wireless communication protocol is an NFC protocol.

11. The glucose monitoring system of claim 8, wherein the reader device is configured to receive the data indicative of the glucose level at a predetermined interval.

12. The glucose monitoring system of claim 11, wherein the predetermined interval is every minute.

13. The glucose monitoring system of claim 1, wherein in the first configuration, the glucose monitoring software application, when executed by the one or more processors, further causes the one or more processors to:
output a graphical user interface (GUI) comprising a first sensor type indicator, wherein the first sensor type indicator comprises a textual description of a brand name associated with the first sensor type.

14. The glucose monitoring system of claim 1, wherein in the second configuration, the glucose monitoring software application, when executed by the one or more processors, further causes the one or more processors to:
output a graphical user interface comprising a second sensor type indicator, wherein the second sensor type indicator comprises a textual description of a brand name associated with the second sensor type.

15. The glucose monitoring system of claim 1, wherein in the first configuration, the glucose monitoring software application, when executed by the one or more processors, further causes the one or more processors to:
cause autonomous communication of the data indicative of the glucose level at a predetermined interval between the sensor control device and the reader device or cause communication of the data indicative of the glucose level according to the first wireless communication protocol; and
in response to a reconnection through the first wireless communication protocol following an interruption of a communication link between the sensor control device and the reader device, the reader device is configured to request historical data indicative of the glucose level from the sensor control device, wherein the historical data indicative of the glucose level is associated with a specific lifecount range; and
upon receiving the request, the sensor control device is further configured to retrieve the requested historical data indicative of the glucose level from a second memory and transmit the requested historical data indicative of the glucose level to the reader device, wherein the reader device is further configured to store the transmitted historical requested historical data indicative of the glucose level in the first memory.

16. The glucose monitoring system of claim 15, wherein the lifecount range includes up to a last eight hours of historical data indicative of the glucose level.

17. The glucose monitoring system of claim 15, wherein the first wireless communication protocol is an NFC protocol.

18. The glucose monitoring system of claim 1, wherein in the second configuration, the glucose monitoring software application, when executed by the one or more processors, further causes the one or more processors to:
cause autonomous communication of the data indicative of the glucose level at a predetermined interval between the sensor control device and the reader device; and
in response to a reconnection following an interruption of a communication link between the sensor control device and the reader device, the reader device is configured to request historical data indicative of the glucose level from the sensor control device according to a lifecount metric; and
upon receiving the request, the sensor control device is further configured to retrieve the requested historical data indicative of the glucose level from a second memory and transmit the requested historical data indicative of the glucose level to the reader device, wherein the reader device is further configured to store the transmitted historical requested historical data indicative of the glucose level in the first memory.

19. The glucose monitoring system of claim 18, wherein the lifecount metric comprises a numerical value configured to be incremented and tracked on the reader device, wherein the lifecount metric is configured to be indicative of an amount of time elapsed since activation of the sensor control device.

20. The glucose monitoring system of claim 18, wherein the data indicative of the glucose level is autonomously communicated between the sensor control device and the reader device according to a second wireless communication protocol.

21. The glucose monitoring system of claim 20, wherein the second wireless communication protocol is a Bluetooth or Bluetooth Low Energy protocol, and wherein the first wireless communication protocol is an NFC protocol.

22. The glucose monitoring system of claim 1, wherein the first configuration is different from the second configuration.

23. The glucose monitoring system of claim 1, wherein the first sensor type is different from the second sensor type.

24. The glucose monitoring system of claim 1, wherein in the first configuration, the glucose monitoring software application, when executed by the one or more processors, further causes the one or more processors to:
output a first set of GUIs corresponding to the first sensor type.

25. The glucose monitoring system of claim 1, wherein in the second configuration, the glucose monitoring software application, when executed by the one or more processors, further causes the one or more processors to:
output a second set of GUIs corresponding to the second sensor type.

26-75. (canceled)
